(19) United States
(12) Patent Application Publication
(10) Pub. No.: US 2004/0229245 A1
(43) Pub. Date: Nov. 18, 2004

(54) Methods and algorithms for performing quality control during gene expression profiling on DNA microarray technology

(76) Inventors: Anton Bittner, Escondido, CA (US); Andrew A. Carmen, San Diego, CA (US); Albert Leung, San Diego, CA (US); Xuejun Liu, San Diego, CA (US); Simon Smith, San Diego, CA (US); Li Sun, Carlsbad, CA (US); Jackson Wan, San Diego, CA (US); Huinian Xiao, San Diego, CA (US); Xiang Yao, San Diego, CA (US); Lynn Yieh, San Diego, CA (US)

Correspondence Address: PHILIP S. JOHNSON, JOHNSON & JOHNSON, ONE JOHNSON & JOHNSON PLAZA, NEW BRUNSWICK, NJ, 08933-7003, US

(21) Appl. No.: 10/753,107

(22) Filed: Jan. 6, 2004

Related U.S. Application Data
(60) Provisional application No. 60/438,002, filed on Jan. 6, 2003.

(51) Int. Cl. C12Q 1/68; G06F 19/00; G01N 33/48; G01N 33/50

(52) U.S. Cl. 435/6; 702/20

(57) ABSTRACT

Methods and algorithms for quality control in gene expression profiling on DNA microarray technology are disclosed including methods and algorithms for assessing quality control of the printing of a DNA microarray, the preparation of an RNA sample, the labeling of probes, the hybridization between a DNA microarray and probes, the result of washing procedures, the result of scanning procedures, and for quantifying the gene expression data produced by imaging the hybridized DNA microarray.

### FIELD OF THE INVENTION

The present invention relates to methods and algorithms for performing quality control measures, and specifically to methods and algorithms for performing quality control measures for gene expression profiling on DNA microarray technology.

### BACKGROUND OF THE INVENTION

The identification and analysis of a particular gene or protein generally has been accomplished by experiments directed specifically towards that gene or protein. With recent advances in the sequencing of the human genome, however, the challenge is to decipher the expression, function, and regulation of thousands of genes, which cannot be realistically accomplished by analyzing one gene or protein at a time. To address this situation, DNA microarray technology has proven to be a valuable tool. By taking advantage of the sequence information obtained from DNA microarrays, the expression and functional relationship of thousands of genes may be resolved.

The expression profiles of thousands of genes have been examined en masse via polynucleotide and oligonucleotide microarrays. Shalon et al., 46 PATHOL. BIOL. 107-09 (1998); Lockhart et al., NUCL. ACIDS SYMP. SER. 11-12 (1998); Schena et al., 16 TRENDS BIOTECHNOL. 301-06 (1998). Several studies have analyzed gene expression profiles in yeast, mammalian cell lines, and disease tissues. Cho et al., 2 MOL. CELL 65-73 (1997); Schena et al., 93 PROC. NATL. ACAD. SCI. USA 10614-19 (1996); Heller at al., 94 PROC. NATL. ACAD. SCI. USA 2150-55 (1997); Welford et al., 26 NUCL. ACIDS RES. 3059-65 (1998).

Microarray technology provides the means to decipher the function of a particular gene based on its expression profile and alteration in its expression levels. In addition, this technology may be used to define the components of cellular pathways as well as the regulation of these cellular components. High-density oligonucleotide and/or polynucleotide microarrays may be used to simultaneously monitor thousands of genes or possibly entire genomes (e.g., Saccharomyces cerevisiae).

Microarrays may also be used for genetic and physical mapping of genomes, DNA sequencing, genetic or disease diagnosis, toxicological studies and genotyping of organisms. For genetic diagnostics, a microarray may contain multiple forms of a mutated gene or multiple genes associated with a particular disease. The microarray may then be probed with DNA or RNA isolated from a patient sample (e.g., blood sample), which may hybridize to one of the mutated or disease genes. Furthermore, microarrays may be used to determine a medical diagnosis. For example, the identity of a pathogenic microorganism may be established unambiguously by hybridizing a patient sample to a microarray containing the genes from many types of known pathogenic DNA. A similar technique may also be used for genotyping of an organism.

Microarrays containing molecular expression markers or predictor genes may be used to conform tissue or cell identifications. In addition, disease progression may be monitored by analyzing the expression patterns of the predictor genes in disease tissues. An alteration in gene expression may be used to define the specific state and stage of the disease. Monitoring the efficacy of certain drug regimens may also be accomplished by analyzing the expression patterns of the predictor genes. For example, decreases or increases in gene expression may be indicative of the efficacy of a particular drug.

Generally, oligonucleotides are used to detect complementary nucleic acid sequences in a particular tissue or cell type. The oligonucleotides may be covalently attached to a support, and arrays of oligonucleotides immobilized on solid supports are used to detect specific nucleic acid sequences. To assess gene expression in a given tissue or cell sample, DNA or RNA is isolated from the tissue or cell, labeled directly or indirectly with a fluorescent dye or other reagent (such as biotin-streptavidin coupled reagents, colloidal suspensions or enzyme coupled reagents), and then hybridized to the microarray. The microarray may contain hundreds to thousands of DNA sequences selected from cDNA libraries, genomic DNA, or expressed sequence tags (ESTs). These sequences may be spotted or synthesized onto the support and then crosslinked to the support by ultraviolet radiation. Following hybridization, the fluorescence intensities (or other measured detection such as electrochemical detection or other forms of spectroscopy not considered fluorescence) of the microarray are analyzed, and these measurements are then used to determine the presence or relative quantity of a particular gene within the sample. This hybridization pattern is used to generate a gene expression profile of the target tissue or cell type.

Thus, differences in gene expression profiles may be used to identify the pathology of many diseases involving alterations of gene expression; that is, normal tissue and diseased tissue may be distinguished by the types of genes and their expression levels. For example, cancer cells evolve from normal cells to highly invasive, metastatic malignancies, which frequently are induced by activation of oncogenes or inactivation of tumor suppressor genes. Differentially expressed sequences can serve as markers or predictors of the transformed state and are, therefore, of potential value in the diagnosis and classification of tumors. The assessment of expression profiles provides meaningful information with respect to tumor type and stage, treatment methods, and prognosis.

### SUMMARY OF THE INVENTION

The present invention relates to methods and algorithms for performing quality control measures, and specifically to methods and algorithms for performing quality control measures for gene expression profiling on microarray technology.

In a preferred embodiment, the present invention may consist of a method of performing quality control in gene expression profiling on a microarray by determining variations between a desired printing and an actual printing of a microarray by retrieving gene expression data from replicate spots, performing a logarithmic transformation on the gene expression data from each of the replicate spots, calculating variations between the log-transformed gene expression data and an expected value for each of the replicate spots, determining a distribution of the variations for each replicate spot, comparing the distribution with a pre-defined distribution, such as a bell curve, and calculating the percentage of the replicate spots for which their variation exceeds a threshold.

In a preferred embodiment, the present invention may consist of a method of performing quality control in gene expression profiling on a microarray during target sample preparation from a biological sample by generating dynamic ranges of values for a target sample and internal spiked controls, calculating a ratio between the dynamic range for the target sample and the dynamic range for the internal spiked controls, and comparing the ratio to a pre-defined value. In a particular embodiment, the dynamic range for the target sample may be computed by performing a logarithmic transformation of the gene expression intensity data for all spots on a microarray, calculating the mean and the standard deviation of the log-transformed data, converting each log-transformed data value into a Z-score value, determining a set of percentiles of Z-score values, and subtracting a minimum value from a maximum value. In a preferred embodiment, the dynamic range for the target sample may be computed with the minimum value equal to the 10^{th} percentile of Z-score values and the maximum value equal to the 90^{th} percentile of Z-score values. In a particular embodiment, the dynamic range for the internal spiked controls may be computed by calculating the median for the high end of the internal spiked controls and the median for the low end of the internal spiked controls, performing a logarithmic transformation for both medians, and subtracting the log-transformed low median value from the log-transformed high median value.

In a preferred embodiment, the present invention may consist of a method of performing quality control in gene expression profiling on a microarray during labeling of target samples by generating a dynamic ranges of values for a labeled target sample, internal spiked controls, and external spiked controls; calculating a first ratio between the dynamic range for the labeled target sample and the dynamic range for the internal spiked controls, comparing the first ratio to a first pre-defined value, calculating a second ratio between the dynamic range of the internal spiked controls and the dynamic range of the external spiked controls, and comparing the second ratio to a second pre-defined value. In a particular embodiment, the dynamic range for the external spiked controls may be computed by calculating the median for the high end of the external spiked controls and the median for the low end of the external spiked controls, performing a logarithmic transformation for both medians, and subtracting the log-transformed low median value from the log-transformed high median value.

In a preferred embodiment, the present invention may consist of a method of performing quality control in gene expression profiling on a microarray during hybridization of the microarray by generating a dynamic ranges of values for a labeled target sample, internal spiked controls, and external spiked controls; calculating a first ratio between the dynamic range for the labeled target sample and the dynamic range for the internal spiked controls; comparing the first ratio to a pre-defined value; calculating a second ratio between the dynamic range of the internal spiked controls and the dynamic range of the external spiked controls; and comparing the second ratio to a pre-defined value. In a particular embodiment, an internal spiked control error flag is set when the first ratio is substantially equal to the first pre-defined value and the second ratio is less than the second pre-defined value. In a particular embodiment, an external spiked control error flag is set when the first ratio is greater than the first pre-defined value, and the second ratio is greater than the second pre-defined value.

In a preferred embodiment, the present invention may also consist of a method of performing quality control in gene expression profiling on a microarray during a washing step of the microarray hybridized with a labeled target sample by retrieving intensity data from one or more replicate spots, calculating a mean intensity from the intensity data, calculating a standard deviation of the intensity data, generating a Z-score transformation for the intensity data from each replicate spot, and calculating the percentage of spots for which the Z-score transformed value exceeds one or more thresholds. In a particular embodiment, three thresholds may be used, such as 1, 2, and 3. In a preferred embodiment, the Z-score transformation is performed by subtracting the mean intensity from the intensity of a particular spot and dividing by the standard deviation of the intensity.

In a preferred embodiment, the present invention may consist of a method of performing quality control in gene expression profiling on a microarray during scanning of the microarray hybridized with a labeled target sample by performing the steps of slide flipping and grid placement. In a preferred embodiment, the step of slide flipping may include retrieving intensity data for one or more replicate spots, comparing intensity data for each replicate spot with a pre-defined normal intensity range, and determining a percentage of replicate spots for which the intensity value is not within a pre-defined normal intensity range. In a preferred embodiment, the step of grid placement may include retrieving intensity data for one or more replicate spots, calculating a first mean and a standard deviation from the intensity data for the replicate spots, calculating one or more second means based on the intensity data for the replicate spots in each row of the oligonucleotides of the microarray, generating a first Z-score transformation for each second mean, calculating one or more third means based on the intensity data for the replicate spots in each column of the microarray, generating a second Z-score transformation for each third mean, and calculating the percentage of first Z-score transformations that exceed a pre-defined value, such as 4, and the percentage of second Z-score transformations that exceed a pre-defined value, such as 4. In a preferred embodiment, the Z-score transformation may be computed by subtracting the first mean from a second mean for a particular row or a third mean for a particular column and dividing by the standard deviation of the log-transformed intensity data.

In a preferred embodiment, the present invention may consist of a method of performing quality control in gene expression profiling on a microarray during quantitation of an image of the microarray hybridized with a labeled target sample by retrieving intensity data for one or more genes wherein each gene contains one or more replicate spots; generating log-transformed intensity data by performing a logarithmic transformation on the intensity data for each replicate spot; retrieving a set of parameters from Imagene for each replicate spot; calculating the mean, the standard deviation, and the CV of the log-transformed intensity data for each gene; and determining outlier spots when the CV for a gene is greater than a pre-defined value, such as 30. In a preferred embodiment, the step of determining outlier spots may consist of calculating one or more metrics for each replicate spot based upon the set of parameters from Imagene and the log-transformed intensity data, computing an outlier score, and marking an replicate spot as an outlier if the outlier score exceeds a pre-defined value, such as 1.

In a preferred embodiment of the present invention, the set of parameters may include, but are not limited to, the mode of the background intensity, the standard deviation of the background intensity, the mean of the background intensity, the mode of the signal intensity, the standard deviation of the signal intensity, the mean of the signal intensity, the median of the signal intensity, the area of the signal intensity, the area of an ignored section, the median of the intensity of the ignored section, and a PositionOff value.

In a preferred embodiment of the present invention, the metrics may include, but are not limited to, a spot intensity ratio equal to the median for the signal intensity of a replicate spot divided by the signal intensity of the replicate spot on a particular DNA microarray; a Z-score transformation of the mode of the background intensity computed by subtracting the mean of the background intensity from the mode of the background intensity and dividing by the standard deviation of the background intensity; a signal CV equal to the standard deviation of the signal intensity divided by the mean of the signal intensity; a background CV equal to the standard deviation of the background intensity divided by the mean of the background intensity; an ignored area ratio computed by dividing the area of the ignored section by the area of the signal intensity; an ignored median ratio equal to the median of the intensity of the ignored section divided by the mode of the signal intensity; a Q signal area value equal to e^{-|A-Ao|/Ao} where Ao is an average of the signal area for one or more genes; and a Z-score transformation of the PositionOff value.

In a preferred embodiment of the present invention, the outlier score for a spot may be computed by setting the outlier score equal to 0; adding a first outlier value, such as 1, to the outlier score when the spot intensity ratio is greater than a pre-defined value, such as 1.4; adding a second outlier value, such as 1, to the outlier score when the spot intensity ratio is less than a pre-defined value, such as 0.714; adding a third outlier value such as 0.5, to the outlier score when the Z-score transformation of the background mode is greater than a third pre-defined value, such as 3; adding a fourth outlier value, such as 1, to the outlier score when the signal CV is greater than a fourth pre-defined value, such as 40, and the logarithmic transformation of the mode of the signal intensity is less than a fifth pre-defined value, such as 3.7; adding a fifth outlier value, such as 0.5, to the outlier score when the background CV is greater than a sixth pre-defined value, such as 40, and the logarithmic transformation of the mode of the signal intensity is less than a seventh pre-defined value, such as 3.7; adding a sixth outlier value, such as 0.5, to the outlier score when the Q signal area is less than an eighth pre-defined value, such as 0.51; adding a seventh outlier value, such as 1, to the outlier score when the ignored median ratio is greater than a ninth pre-defined value, such as 6; and adding an eighth outlier value, such as 0.5, to the outlier score when the Z-score transformation of the PositionOff value is greater than a tenth pre-defined value, such as 5.

### BRIEF DESCRIPTION OF THE DRAWINGS

The accompanying figures, which are incorporated in and constitute a part of the specification, illustrate embodiments of the invention and, together with the description, serve to explain the objects, advantages and principles of the invention.

FIG. 1 is a flow chart of a method of generating gene expression data on a biological chip.

FIG. 2 is a flow chart of a method of generating gene expression data on a biological chip implementing quality control processes of the present invention.

FIG. 3 is a flow chart of a method of collecting data during quality control processes of the present invention.

FIG. 4 is a flow chart of a method of analyzing data during quality control processes of the present invention.

FIG. 5 is a depiction of failure types based upon measurements taken in the course of quality control processes of the present invention.

FIG. 6 is a block diagram that shows the relationship between quality control analyzing modules in the present invention.

### DETAILED DESCRIPTION OF THE INVENTION

It is to be understood that the terminology used herein is for the purpose of describing particular embodiments only, and is not intended to limit the scope of the present invention which will be limited only by the appended claims.

It must be noted that as used herein and in the appended claims, the singular forms “a,” “an,” and “the” include plural references unless the context clearly dictates otherwise. Thus, for example, reference to “a gene” is a reference to one or more genes and includes equivalents thereof known to those skilled in the art, and so forth.

Unless defined otherwise, all technical and scientific terms used herein have the same meaning as commonly understood to one of ordinary skill in the art to which this invention belongs. Although any methods, devices, and materials similar or equivalent to those described herein can be used in the practice or testing of the invention, the preferred methods, devices and materials are now described.

All publications and patents mentioned herein are hereby incorporated herein by reference for the purpose of describing and disclosing, for example, the methodologies that are described in the publications which might be used in connection with the present invention. Publications discussed throughout the text are provided solely for their disclosure prior to the filing date of the present application. Nothing herein is to be construed as an admission that the inventors are not entitled to antedate such disclosure by virtue of prior invention.

### Definitions

For convenience, the meaning of certain terms and phrases employed in the specification, examples, and appended claims are provided below. The definitions are not meant to be limiting in nature and serve to provide a clearer understanding of certain aspects of the present invention.

The term “gene” refers to a nucleic acid sequence that comprises control and coding sequences necessary for the production of a polypeptide or precursor. The polypeptide can be encoded by a full length coding sequence or by any portion of the coding sequence. The gene may be derived in whole or in part from any source known to the art, including a plant, a fungus, an animal, a bacterial genome or episome, eukaryotic, nuclear or plasmid DNA, cDNA, viral DNA, or chemically synthesized DNA. A gene may contain one or more modifications in either the coding or the untranslated regions that could affect the biological activity or the chemical structure of the expression product, the rate of expression, or the manner of expression control. Such modifications include, but are not limited to, mutations, insertions, deletions, and substitutions of one or more nucleotides. The gene may constitute an uninterrupted coding sequence or it may include one or more introns, bound by the appropriate splice junctions.

The terms “gene expression profile” or “gene expression signature” refer to one or more genes whose expression pattern is representative of representing a particular cell or tissue type (e.g., neuron, coronary artery endothelium, or disease tissue).

“Gene expression” refers to the process by which a nucleic acid sequence undergoes successful transcription and translation such that detectable levels of the nucleotide sequence are expressed.

The term “upregulated” refers to messenger RNA levels encoding a gene which are detectably increased in a tissue sample from a treated animal compared with the messenger RNA levels encoding the same gene in a tissue sample from an untreated animal.

The term “downregulated” refers to messenger RNA levels encoding a gene which are detectably decreased in a tissue sample from a treated animal compared with the messenger RNA levels encoding the same gene in a tissue sample from an untreated animal.

The term “genome” is intended to include the entire DNA complement of an organism, including the nuclear DNA component, chromosomal or extrachromosomal DNA, as well as the cytoplasmic domain (e.g., mitochondrial DNA).

A “polynucleotide” or “oligonucleotide” refers to a chain of nucleotides. Preferably, the chain has from about 5 to about 10,000 nucleotides, more preferably from about 50 to 1,000 nucleotides. The term “polynucleotide target” refers to a polynucleotide sequence capable of hybridizing with a “polynucleotide probe” to form a polynucleotide target/probe complex under hybridization conditions. In some instances, the sequences will be complementary (no mismatches) when aligned. In other instances, there may be a substantial mismatch, up to 10%. The oligonucleotide may be a naturally occurring oligonucleotide or a synthetic oligonucleotide. Oligonucleotides may be prepared by the phosphoramidite method (Beaucage and Carruthers, 22 TETRAHEDRON LETT. 1859-62 (1981)), or by the triester method (Matteucci et al., 103 J. AM. CHEM. SOC. 3185 (1981)), or by other chemical methods known in the art.

A “fragment” refers to a sequence which is a portion of a polynucleotide target sequence.

The terms “array” and “microarray” refer to the type of genes represented on an array by oligonucleotides, and where the type of genes represented on the array is dependent on the intended purpose of the array (e.g., to monitor expression of human genes). The oligonucleotides on a given array may correspond to the same type, category, or group of genes. Genes may be considered to be of the same type if they share some common characteristic such as species of origin (e.g., human, mouse, rat); disease state (e.g., cancer); functions (e.g., protein kinases, tumor suppressors); or biological process (e.g., apoptosis, signal transduction, cell cycle regulation, proliferation, differentiation). For example, one array type may be a “cancer array” in which each of the array oligonucleotides corresponds to a gene associated with a cancer. An “epithelial array” may be an array of oligonucleotides corresponding to unique epithelial genes. Similarly, a “cell cycle array” may be an array type in which the oligonucleotides correspond to unique genes associated with the cell cycle. The terms “DNA chip,” “chip,” “DNA array,” “DNA microarray,” “array,” and “microarray” are intended to be interchangeable.

The microarrays on which the present inventive method takes place may comprise from about 100 to about 1,000,000 or more different oligonucleotide or polynucleotide probes. In a particular embodiment, the oligonucleotide probes may range from about 10 to about 1000 nucleotides in length. Furthermore, the hybridization signal from each of the array elements is preferably individually distinguishable. In a preferred embodiment, the array elements comprise polynucleotide sequences.

The term “cell type” refers to a cell from a given source (e.g., a tissue or an organ), or a cell in a given state of differentiation, or a cell associated with a given pathology or genetic makeup.

The term “activation” as used herein refers to any alteration of a signaling pathway or biological response including, for example, increases above basal levels, restoration to basal levels from an inhibited state, and stimulation of the pathway above basal levels.

The term “differential expression” refers to both quantitative as well as qualitative differences in the temporal and tissue expression patterns of a gene. For example, a differentially expressed gene may have its expression activated or completely inactivated in normal versus disease conditions. Such a qualitatively regulated gene may exhibit an expression pattern within a given tissue or cell type that is detectable in either control or disease conditions, but is not detectable in both.

The term “detectable” refers to an RNA expression pattern which is detectable via the standard techniques of polymerase chain reaction (PCR), reverse transcriptase-(RT) PCR, differential display, and Northern analyses, which are well known to those of skill in the art.

A “target gene” refers to a nucleic acid, often derived from a biological sample, to which an oligonucleotide (or PCR product) probe is designed to specifically hybridize. It is either the presence or absence of the target nucleic acid that is to be detected, or the amount of the target nucleic acid that is to be quantified. The target nucleic acid has a sequence that is complementary to the nucleic acid sequence of the corresponding probe directed to the target. The target nucleic acid may also refer to the specific subsequence of a larger nucleic acid to which the probe is directed or to the overall sequence (e.g., gene or mRNA) whose expression level it is desired to detect.

The term “complementary” refers to the topological compatibility or matching together of interacting surfaces of a probe molecule and its target. The target and its probe can be described as complementary, and furthermore, the contact surface characteristics are complementary to each other. Hybridization or base pairing between nucleotides or nucleic acids, such as, for example, between the two strands of a double-stranded DNA molecule or between an oligonucleotide probe and a target are complementary.

The term “hybridization” refers to the binding, duplexing, or hybridizing of a nucleic acid molecule to a particular nucleic acid sequence under stringent conditions.

The term “stringent conditions” refers to conditions under which a target probe may hybridize to a complementary oligonucleotide sequence on a microarray, but to no other sequences. Stringent conditions are sequence-dependent (e.g., longer sequences hybridize specifically at higher temperatures). Generally, stringent conditions are selected to be about 5° C. lower than the thermal melting point (Tm) for the specific sequence at a defined ionic strength and pH. The Tm is the temperature (under defined ionic strength, pH, and nucleic acid concentration) at which 50% of the probes complementary to the target sequence hybridize to the target sequence at equilibrium. Typically, stringent conditions will be those in which the salt concentration is at least about 0.01 M to about 1.0 M sodium ion concentration (or other salts) at about pH 7.0 to about pH 8.3 and the temperature is at least about 30° C. for short probes (e.g., 10 to 50 nucleotides). Stringent conditions may also be achieved with the addition of destabilizing agents such as formamide.

The term “label” refers to agents that are capable of providing a detectable signal, either directly or through interaction with one or more additional members of a signal producing system and are attachable to target sample probes of the present invention. Labels that are directly detectable and may find use in the present invention include: fluorescent labels, where the wavelength of light absorbed by the fluorophore may generally range from about 300 nm to about 900 nm, usually from about 400 nm to about 800 nm, and where the absorbance maximum may typically occur at a wavelength ranging from about 500 nm to about 800 nm. Specific fluorophores for use in singly labeled primers include: fluorescein, rhodamine, BODIPY, cyanine dyes and the like. Radioactive isotopes, such as ³⁵S, ³²P, ³H, and the like may also be utilized as labels. Examples of labels that provide a detectable signal through interaction with one or more additional members of a signal producing system include capture moieties that specifically bind to complementary binding pair members, where the complementary binding pair members comprise a directly detectable label moiety, such as a fluorescent moiety as described above. The label should be such that it does not provide a variable signal, but instead provides a constant and reproducible signal over a given period of time. Capture moieties of interest include ligands (e.g., biotin) where the other member of the signal producing system could be fluorescently-labeled streptavidin, and the like. The target molecules may be end-labeled, i.e., the label moiety is present at a region at least proximal to, and preferably at, the 5′ terminus of the target.

The term “protecting group” as used herein refers to any of the groups which are designed to block one reactive site in a molecule while a chemical reaction is carried out at another reactive site. The proper selection of protecting groups for a particular synthesis may be governed by the overall methods employed in the synthesis. For example, in photolithography synthesis, discussed below, the protecting groups are photolabile protecting groups such as NVOC and MeNPOC. In other methods, protecting groups may be removed by chemical methods and include groups such as FMOC, DMT, and others known to those of skill in the art.

The terms “support” and “substrate” include material having a rigid or semi-rigid surface. Such materials may preferably take the form of plates or slides, small beads, pellets, disks or other convenient forms, and may comprise glass, silicone, polymers, gels, matricies, or any substance capable of supporting polynucleotide or oligonucleotide sequences of the arrays of the present invention, although other forms may be used. In some embodiments, at least one surface of the substrate will be substantially flat. In other embodiments, a roughly spherical shape or other matrix may be preferred.

As mentioned above, the microarray may be present on a rigid substrate. By rigid, the support is solid and preferably does not readily bend. As such, the rigid substrates of the microarrays are sufficient to provide physical support and structure to the oligonucleotide probes present thereon under the assay conditions in which the microarray is utilized, particularly under high-throughput handling conditions.

The term “spatially directed oligonucleotide synthesis” refers to any method of directing the synthesis of an oligonucleotide to a specific location on a substrate.

The term “background” refers to hybridization signals resulting from non-specific binding, or other interactions, between the labeled target nucleic acids and components of the oligonucleotide microarray (e.g., the oligonucleotides, control oligonucleotides, the array substrate). Background signals may also be produced by intrinsic fluorescence of the microarray components themselves. A single background signal can be calculated for the entire array, or a different background signal may be calculated for each target nucleic acid. The background may be calculated as the average hybridization signal intensity, where a different background signal is calculated for each target gene. Alternatively, background may be calculated as the average hybridization signal intensity produced by hybridization to oligonucleotides that are not complementary to any sequence found in the sample (e.g., probes directed to nucleic acids of the opposite sense or to genes not found in the sample such as bacterial genes where the sample is mammalian nucleic acids). The background can also be calculated as the average signal intensity produced by regions of the array that lack any oligonucleotides at all.

The term “bead” refers to supports for use with the present invention. Such beads may have a wide variety of forms, including microparticles, beads, membranes, slides, plates, micromachined chips, and the like. Likewise, substrates or supports of the invention may comprise a wide variety of compositions, including glass, gels, polymers, plastic, silicon, alkanethiolate-derivatized gold, cellulose, low crosslinked and high crosslinked polystyrene, silica gel, polyamide, and the like. Other materials and shapes may be used, including pellets, disks, capillaries, hollow fibers, needles, solid fibers, cellulose beads, matrices, pore-glass beads, silica gels, polystyrene beads optionally crosslinked with divinylbenzene, grafted co-poly beads, poly-acrylamide beads, latex beads, dimethylacrylamide beads optionally crosslinked with N,N-bis-acryloyl ethylene diamine, and glass particles coated with a hydrophobic polymer.

The term “biological sample” refers to a sample obtained from an organism (e.g., patient) or from components (e.g., cells) of an organism. The sample may be of any biological tissue or fluid. The sample may be a “clinical sample,” which is a sample derived from a patient. Such samples include, but are not limited to, sputum, blood, blood cells (e.g., white cells), amniotic fluid, plasma, semen, bone marrow, tissue or fine needle biopsy samples, urine, peritoneal fluid, and pleural fluid, or cells therefrom. Biological samples may also include sections of tissues such as frozen sections taken for histological purposes.

The terms “Quality Control” or “QC” refer to a process to assess the successful rate and reproducibility in generating gene expression data using DNA chip (microarray) technology.

The term “QC Data Collector” refers to a collection of designs in experimental procedures and chip layout used to collect essential control information related to key elements of the whole process of generating gene expression data. Such key elements include RNA preparation, probe labeling, hybridization, washing, scanning, and chip printing.

The term “QC Data Analyzer” refers to a collection of algorithms used to analyze the quality control information collected by the QC Data Collector and, in one embodiment, to produce a management report to summarize Quality Control information.

This process and algorithms of the present invention are applicable to arrays composed of oligonucleotide, polynucleotide, peptide, protein or other materials which utilize a hybridization and/or binding of a second labeled moiety, which subsequently requires a procedure for image acquisition and analysis as a procedure for quality control in a similar fashion. The array of the present invention may be spotted on glass or any flat surface, by spotting with pens, inkjet, light/photo directed synthesis, piezo-electric procedures, electrochemical directed synthesis or other procedures. The minimum number of spots or features required for this procedure will typically be greater than 50 total spots per array, with no maximum limit. The exact gene products used for the algorithms of the present invention are not essential to this invention and assert that any unrelated gene product to the organism being tested will suffice for these procedures for spiked in controls and negative controls. The exact positive controls and housekeeping genes used in this invention are not specific to this invention, any similar gene used in an organism specific manner are sufficient. The exact numbers of copies of all controls on the chips and/or concentrations of spiked in controls are not essential to this invention as any number of similar conditions can employ this algorithm.

Reference will now be made in detail to implementations of the present invention as illustrated in the accompanying drawings. Wherever possible, the same reference numbers will be used throughout the drawings and the following description to refer to the same or like parts.

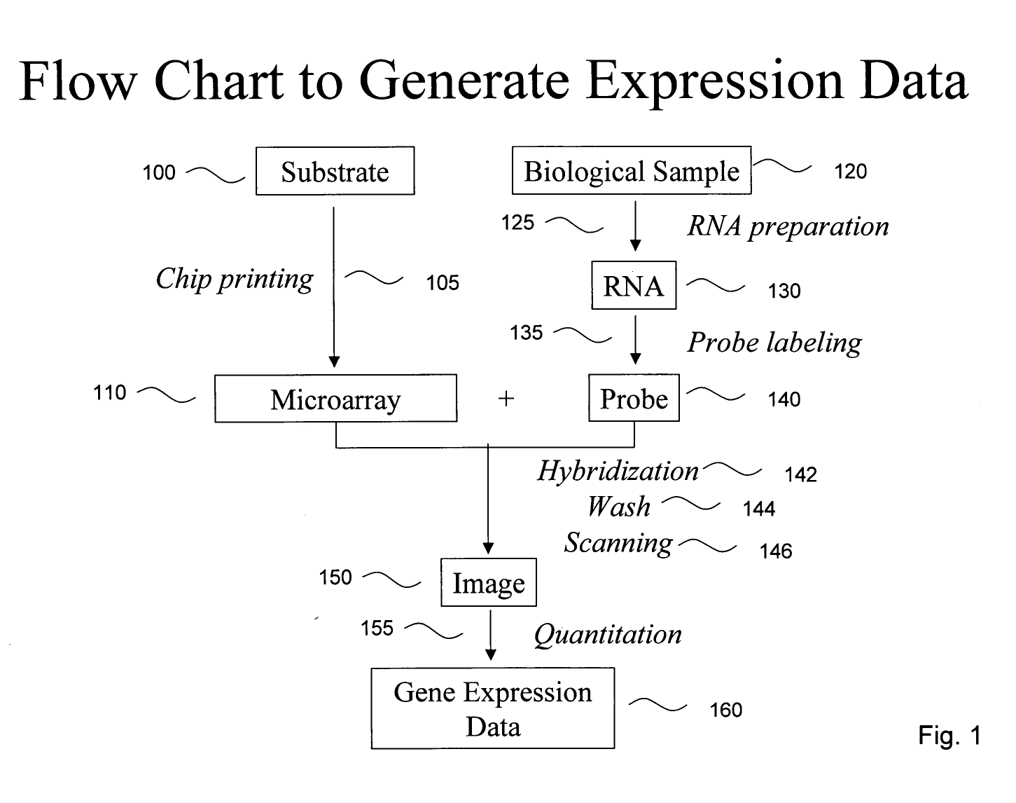

FIG. 1 depicts a flow chart for microarray (DNA chip) analysis and associated quality control processes, which are the subject of the present invention. In microarray analysis, nucleic acids (e.g., RNA 130) may be isolated from a biological sample 120. Nucleic acid samples include, but are not limited to, mRNA transcripts of the gene or genes, cDNA reverse transcribed from the mRNA, cRNA transcribed from the cDNA, DNA amplified from the genes, RNA transcribed from the amplified DNA, and the like.

A microarray 110 may contain any number of different target oligonucleotides. A microarray may have from about 2 to about 1,000,000 probes. In addition, the microarray may have a density of more than 100 to more than 10,000 oligonucleotides at known locations per cm². The microarrays may be produced through spatially directed oligonucleotide synthesis. Methods for spatially directed oligonucleotide synthesis include, without limitation, light-directed and/or electrochemically driven oligonucleotide synthesis, microlithography, application by ink jet, microchannel deposition to specific locations and sequestration with physical barriers. In general these methods involve generating active sites, usually by removing protective groups; and coupling to the active site a nucleotide, which, itself, optionally has a protected active site if further nucleotide coupling is desired.

A microarray 110 may be configured, for example, by in situ synthesis or by direct deposition (“spotting” or “printing”) of synthesized oligonucleotides onto the support. The oligonucleotide probes are used to detect complementary nucleic acid sequences in a sample of interest. In situ synthesis has several advantages over direct placement such as higher yields and consistency, efficiency, cost, and potential use of combinatorial strategies. Southern et al. (1999). However, for longer nucleic acid sequences such as PCR products, deposition may be the preferred method. Generation of microarrays by in situ synthesis may be accomplished by a number of methods including photochemical deprotection, ink-jet delivery, and flooding channels. Lipshutz et al., 21 NATURE GENET. 20-24 (1999); Blanchard et al., 11 BIOSENSORS AND BIOELECTRONICS 687-90 (1996); Maskos et al., 21 NUCL. ACIDS RES. 4663-69 (1993).

A number of different microarray configurations and methods for their production are known to those of skill in the art and are disclosed in U.S. Pat. Nos. 5,242,974; 5,384,261; 5,405,783; 5,412,087; 5,424,186; 5,429,807; 5,436,327; 5,445,934; 5,556,752; 5,405,783; 5,412,087; 5,424,186; 5,429,807; 5,436,327; 5,472,672; 5,527,681; 5,529,756; 5,545,531; 5,554,501; 5,561,071; 5,571,639; 5,593,839; 5,624,711; 5,700,637; 5,744,305; 5,770,456; 5,770,722; 5,837,832; 5,856,101; 5,874,219; 5,885,837; 5,919,523; 6,093,302, 6,280,595, 6,022,963; 6,077,674; and 6,156,501; the disclosures of which are herein incorporated by reference. Patents describing methods of using arrays in various applications include: U.S. Pat. Nos. 5,143,854; 5,288,644; 5,324,633; 5,432,049; 5,470,710; 5,492,806; 5,503,980; 5,510,270; 5,525,464; 5,547,839; 5,580,732; 5,661,028; 5,848,659; and 5,874,219; the disclosures of which are herein incorporated by reference.

To detect gene expression, oligonucleotides may be designed and synthesized based on known sequence information. For example, 20- to 30-mer oligonucleotides, which may be derived from known cDNA or EST sequences, may be selected to monitor expression. Lipshutz et al. (1999). The oligonucleotides may be selected from a number of sources including nucleic acid databases such as GenBank, Unigen, HomoloGene, RefSeq, dbEST, and dbSNP. Wheeler et al., 29 NUCL. ACIDS RES. 11-16 (2001). Generally, the oligonucleotide is complementary to the reference sequence, preferably unique to the tissue or cell type (e.g., skeletal muscle, neuronal tissue) of interest, and preferably hybridizes with high affinity and specificity. Lockhart et al., 14 NATURE BIOTECHNOL. 1675-80 (1996). In addition, the oligonucleotides may represent non-overlapping sequences of the reference sequence which improves redundancy resulting in a reduction in false positive rate and an increased accuracy in target quantitation. Lipshutz et al. (1999).

The selected polynucleotide sequences can be manipulated further to optimize the performance of the polynucleotide sequences as hybridization sequences. Some sequences may not hybridize effectively under hybridization conditions due to secondary structure. To optimize probe hybridization, the probe sequences are examined using a computer algorithm to identify portions of genes without potential secondary structure. Such computer algorithms are well known in the art, such as OLIGO 4.06 Primer Analysis Software (National Biosciences) or LASERGENE software (DNASTAR). These programs can search nucleotide sequences to identify stem loop structures and tandem repeats and to analyze G+C content of the sequence (those sequences with a G+C content greater than 60% are excluded). Alternatively, the sequences can be optimized by trial and error. Experiments can be performed to determine whether sequences and complementary target polynucleotides hybridize optimally under experimental conditions.

The polynucleotide sequences can be any RNA-like or DNA-like material, such as mRNAs, cDNAs, genomic DNA, peptide nucleic acids, branched DNAs and the like. The polynucleotide sequences can be in sense or antisense orientations.

In one embodiment, the polynucleotide sequences are cDNAs. The size of the DNA sequence of interest may vary, and is preferably from about 50 to about 10,000 nucleotides, more preferably from about 150 to about 3,500 nucleotides. In a second embodiment, the polynucleotide sequences are vector DNAs. In this case the size of the DNA sequence of interest, i.e., the insert sequence, may vary from about 50 to about 10,000 nucleotides, more preferably from about 150 to about 3,500 nucleotides.

The polynucleotide sequences can be prepared by a variety of synthetic or enzymatic schemes which are well known in the art. Caruthers et al., 7 NUCL. ACIDS SYMP. SER. 215-23 (1980). Nucleotide analogues can be incorporated into the polynucleotide sequences by methods well known in the art. The only requirement is that the incorporated nucleotide analogues must serve to base pair with polynucleotide target probe sequences. For example, certain guanine nucleotides can be substituted with hypoxanthine which base pairs with cytosine residues. However, these base pairs are less stable than those between guanine and cytosine. Alternatively, adenine nucleotides can be substituted with 2,6-diaminopurine which can form stronger base pairs than those between adenine and thymidine. Additionally, the polynucleotide sequences can include nucleotides that have been derivatized chemically or enzymatically. Typical modifications include derivatization with acyl, alkyl, aryl or amino groups.

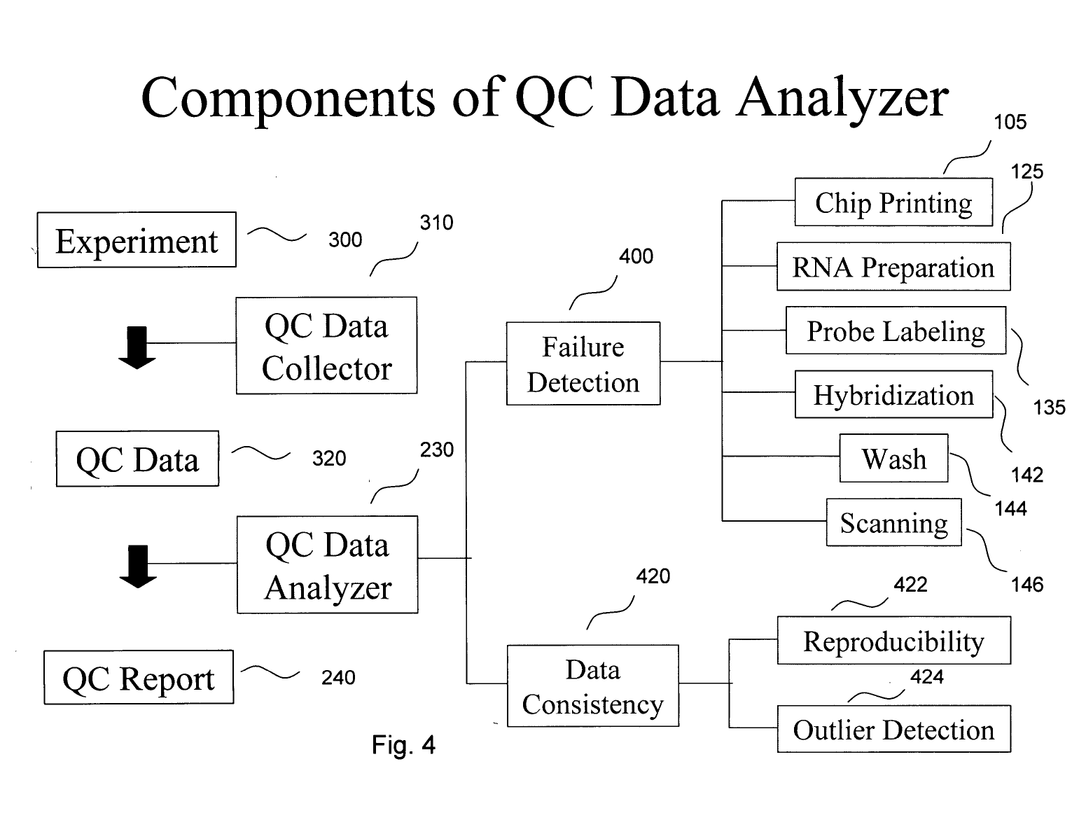

Referring to FIG. 1, the present invention relates to methods of performing quality control over the process of configuring a microarray 110 from a substrate 100. Specifically, a chip printing 105 quality control process may be performed to determine if the microarray 110 has been properly formed. The chip printing 105 quality control process is described in more detail in reference to FIG. 4.

Microarrays 110 may have a plurality of modified oligonucleotides or polynucleotides stably associated with the surface of a support, e.g., covalently attached to the surface with or without a linker molecule. Each oligonucleotide on the array 110 comprises a modified oligonucleotide composition of known identity and usually of known sequence. By stable association, the associated modified oligonucleotides maintain their position relative to the support 100 under hybridization and washing conditions.

The oligonucleotides may be non-covalently or covalently associated with the support surface. Examples of non-covalent association include non-specific adsorption, binding based on electrostatic interactions (e.g., ion pair interactions), hydrophobic interactions, hydrogen bonding interactions, and specific binding through a specific binding pair member covalently attached to the support surface. Examples of covalent binding include covalent bonds formed between the oligonucleotides and a functional group present on the surface of the rigid support (e.g., —OH), where the functional group may be naturally occurring or present as a member of an introduced linking group.

The support or substrate 100 may comprise one or a number of materials, including glass. There are several advantages for utilizing glass supports 100 in constructing a microarray. For example, microarrays prepared using a glass support 100 generally utilize microscope slides due to the low inherent fluorescence, thus, minimizing background noise. Moreover, hundreds to thousands of oligonucleotide probes may be attached to a slide 100. The glass slides 100 may be coated with polylysine, amino silanes, or amino-reactive silanes which enhance the hydrophobicity of the slide and improves the adherence of the oligonucleotides. Duggan et al. (1999). Ultraviolet irradiation is used to crosslink the oligonucleotide probes to the glass support 100. Following irradiation, the support 100 may be treated with succinic anhydride to reduce the positive charge of the amines. For double-stranded oligonucleotides, the support 100 may be subjected to heat (e.g., 95° C.) or alkali treatment to generate single-stranded probes. An additional advantage to using glass is its nonporous nature, thus requiring a minimal volume of hybridization buffer resulting in enhanced binding of target samples to probes.

The oligonucleotides of a microarray 110 may be arranged on the surface of the support based on size. With respect to the arrangement according to size, the probes may be arranged in a continuous or discontinuous size format. By continuous, each successive position in the microarray 110, for example, a successive position in a lane of probes, comprises oligonucleotide probes of the same molecular weight. By discontinuous format, each position in the pattern (e.g., band in a lane) represents a fraction of target molecules derived from the original source, where the probes in each fraction will have a molecular weight within a determined range.

The array is preferably organized in an ordered fashion so that each oligonucleotide is present at a specified location on the substrate. Because the oligonucleotides are at specified locations on the substrate, the hybridization patterns and intensities (which together create a unique expression profile) can be interpreted in terms of expression levels of particular genes.

Each microarray 110 may contain oligonucleotides isolated from the same source (e.g., the same tissue), or from different sources (e.g., different tissues, different species, disease and normal tissue). As such, oligonucleotides isolated from the same source may be represented by one or more lanes or areas; whereas oligonucleotides from different sources may be represented by individual patterns on the microarray where oligonucleotides from the same source are similarly located. Therefore, the surface of the support may represent a plurality of patterns of oligonucleotides derived from different sources (e.g., tissues), where the probes in each lane are arranged according to size, either continuously or discontinuously.

Generally, the oligonucleotides are generated by standard synthesis chemistries such as phosphoramidite chemistry. U.S. Pat. Nos. 4,980,460; 4,725,677; 4,415,732; 4,458,066; and 4,973,679; Beaucage and Iyer, 48 TETRAHEDRON 2223-2311 (1992). Alternative chemistries which create non-natural backbone groups, such as phosphorothionate and phosphoroamidate may also be employed.

Control oligonucleotides corresponding to genomic DNA, housekeeping genes, or negative and positive control genes may also be present on the microarray. These oligonucleotides are used to calibrate background or basal level of expression or provide other useful information. These oligonucleotides may range from about 5 to about 50 nucleotides, or more.

Such controls may include oligonucleotides that are perfectly complementary to labeled reference oligonucleotide probes that are added to the target sample probes. The signals obtained from these controls after hybridization provide a control for variations in hybridization conditions, label intensity, efficiency, and other factors that may cause the hybridization signal to vary between microarrays. To normalize fluorescence intensity measurements, for example, signals from all probes of the microarray may be divided by the signal from the control probes.

Other controls comprise oligonucleotides that hybridize specifically with constitutively expressed genes in the target sample and are designed to control for the overall metabolic activity of a cell. Analysis of the variations in the expression levels of these controls as compared to the expression level of the target nucleic acid indicates whether variations in expression level of a gene is due specifically to changes in transcription rate of that gene or to general variations in the source of the sample. Thus, if the expression levels of both the expression control and the target gene decrease or increase, these alterations may be attributed to changes in the metabolic activity of the source of the sample as a whole, not to differential expression of the target gene in question. However, if only the expression of the target gene varies, then the variation in the expression may be attributed to differences in regulation of that gene and not to overall variations in the metabolic activity of the source of the sample. Constitutively expressed genes such as housekeeping genes (e.g., 13-actin gene, transferrin receptor gene, GAPDH gene) may serve as expression level controls.

Yet other controls may also be used for expression level controls or for normalization controls. These oligonucleotides provide a control for non-specific binding or cross-hybridization to a nucleic acid in the sample other than the target to which the oligonucleotide is directed. Mismatch controls are oligonucleotides identical to the corresponding test or control probes except for the presence of one or more mismatched bases. One or more mismatches (e.g., substituting guanine, cytidine, or thymine for adenine) are selected such that under appropriate hybridization conditions (e.g., stringent conditions), the test or control oligonucleotides would be expected to hybridize with its target sequence, but the mismatch oligonucleotides would not hybridize or would hybridize to a significantly lesser extent.

Surfaces of the support 100 may be composed of the same material as the support 100. Alternatively, the surface may be composed of any of a wide variety of materials, for example, polymers, gels, plastics, resins, polysaccharides, silica or silica-based materials, carbon, metals, inorganic glasses, membranes, or any of the above-listed substrate materials. Preferably, the surface may contain reactive groups, such as carboxyl, amino, or hydroxyl groups. Most preferably, the surface is optically transparent and will have surface SiOH functionalities, such as are found on silica surfaces.

The surface of the support 100 may possess a layer of linker molecules (or spacers). The linker molecules are preferably of sufficient length to permit the oligonucleotide probes on the support 100 to hybridize to nucleic acid molecules and to interact freely with molecules exposed to the support 100. In one embodiment, the linker molecules may be about 6-50 molecules long to provide sufficient exposure. The linker molecules may also be, for example, aryl acetylene, ethylene glycol oligomers containing about 2-10 monomer units, diamines, diacids, amino acids, or combinations thereof.

The linker molecules may be attached to the support 100 via carbon-carbon bonds using, for example, (poly)trifluorochloroethylene surfaces, or preferably, by siloxane bonds (using, for example, glass or silicon oxide surfaces). Siloxane bonds may be formed via reactions of linker molecules containing trichlorosilyl or trialkoxysilyl groups. The linker molecules may also have a site for attachment of longer chain portion. For example, groups that are suitable for attachment to a longer chain portion may include amines, hydroxyl, thiol, and carboxyl groups. The surface attaching portions may include aminoalkylsilanes, hydroxyalkylsilanes, bis(2-hydroxyethyl)-aminopropyltriethoxysilane, 2-hydroxyethylaminopropyltriethoxysilane, aminopropyltriethoxysilane, and hydroxypropyltriethoxysilane. The linker molecules may be attached in an ordered array (e.g., as parts of the head groups in a polymerized Langinuir Blodgett film). Alternatively, the linker molecules may be adsorbed to the surface of the support 100.

Typically, the length of the linker may be a length which is at least the length spanned by, for example, two to four nucleotide monomers. The linking group may be an alkylene group (from about 6 to about 24 carbons in length), a polyethyleneglycol group (from about 2 to about 24 monomers in a linear configuration), a polyalcohol group, a polyamine group (e.g., spermine, spermidine, or polymeric derivatives thereof), a polyester group (e.g., poly(ethylacrylate) from about 3 to about 15 ethyl acrylate monomers in a linear configuration), a polyphosphodiester group, or a polynucleotide (from about 2 to about 12 nucleic acids). For in situ synthesis, the linking group may be provided with functional groups which can be suitably protected or activated. The linking group may be covalently attached to the oligonucleotides by an ether, ester, carbamate, phosphate ester, or amine linkage. Preferred linkages are phosphate ester linkages which can be formed in the same manner as the oligonucleotide linkages. For example, hexaethyleneglycol may be protected on one terminus with a photolabile protecting group (e.g., NVOC or MeNPOC) and activated on the other terminus with 2-cyanoethyl-N,N-diisopropylamino-chlorophosphite to form a phosphoramidite. This linking group may then be used for construction of oligonucleotide probes in the same manner as the photolabile-protected, phosphoramidite-activated nucleotides.

Furthermore, the linker molecules and oligonucleotides may contain a functional group with a bound protective group. Preferably, the protective group is on the distal or terminal end of the linker molecule opposite the support. The protective group may be either a negative protective group (e.g., the protective group renders the linker molecules less reactive with a monomer upon exposure) or a positive protective group (e.g., the protective group renders the linker molecules more reactive with a monomer upon exposure). In the case of negative protective groups, an additional step of reactivation may be required. This may be done by heating. The protective group on the linker molecules may be selected from a wide variety of positive light-reactive groups preferably including nitro aromatic compounds, such as o-nitrobenzyl derivatives or benzylsulfonyl. Other protective groups include 6-nitroveratryloxycarbonyl (NVOC), 2-nitrobenzyloxycarbonyl (NBOC) or α,α-dimethyl-dimethoxybenzyloxycarbonyl (DDZ). Photoremovable protective groups are described in, for example, Patchornik, 92 J. AM. CHEM. SOC. 6333 (1970) and Amit et al., 39 J. ORG. CHEM. 192 (1974).

The present invention relates to methods for performing quality control over the process of preparing target sample probes from nucleic acids, such as RNA 130, from a biological sample 120. Specifically, an RNA preparation 125 quality control process may be performed to determine if the target sample probe 130 has been properly formed. The RNA preparation 125 quality control process is described in more detail in reference to FIG. 4.

Notably, target sample probes 130 may be generated from total RNA, isolated from a specific tissue or cell type, by dT-primed reverse transcription producing cDNA. See, e.g., SAMBROOK ET AL., MOLECULAR CLONING: A LABORATORY MANUAL, Cold Spring Harbor Press, New York (1989); AUSBEL ET AL., CURRENT PROTOCOLS IN MOLECULAR BIOLOGY, John Wiley & Sons, Inc. (1995). The cDNA may then be transcribed to cRNA by in vitro transcription resulting in a linear amplification of the RNA. The target samples can be any sample comprising polynucleotide probes and obtained from any bodily fluid (blood, urine, saliva, phlegm, gastric juices, etc.), cultured cells, biopsies, or other tissue preparations. The target samples can be derived from humans or animal models.

DNA or RNA can be isolated from the sample according to any of a number of methods well known to those of skill in the art. For example, methods of purification of nucleic acids are described in Laboratory Techniques in Biochemistry and Molecular Biology: Hybridization With Nucleic Acid Probes, Part I. Theory and Nucleic Acid Preparation, P. Tijssen, ed. Elsevier (1993). In one preferred embodiment, total RNA is isolated using the TRIZOL® total RNA isolation reagent (Life Technologies, Inc.) and mRNA is isolated using oligo d(T) column chromatography or glass beads. When polynucleotide probes are amplified it is desirable to amplify the nucleic acid sample and maintain the relative abundances of the original sample, including low abundance transcripts. RNA can be amplified in vitro, in situ or in vivo. See U.S. Pat. No. 5,514,545.

Amplification methods include, but are not limited to, PCR (Innis et al., PCR PROTOCOLS. A GUIDE TO METHODS AND APPLICATION, Academic Press, Inc. San Diego, (1990)), ligase chain reaction (LCR) (Wu and Wallace, 4 GENOMES 560 (1989); Landegren et al., 241 SCIENCE 1077 (1988); and Barringer et al., 89 GENE 117 (1990)), transcription amplification (Kwoh et al., 86 PROC. NATL. ACAD. SCI. USA 1173 (1989)), and self-sustained sequence replication (Guatelli et al., 87 PROC. NATL. ACAD. SCI. USA 1874 (1990)). Prior to hybridization, it may be desirable to fragment the polynucleotide target sample probes. Fragmentation improves hybridization by minimizing secondary structure and cross-hybridization to other polynucleotide probes in the sample or to noncomplementary polynucleotide sequences. Fragmentation may be performed by means known to those skilled in the art. Such means include mechanical and chemical means.

The present invention relates to methods for performing quality control over the process of labeling the target samples 130 to produce labeled target sample probes 140. Specifically, a probe labeling 135 quality control process may be performed to determine if the labeled probes 140 have been properly constructed. The probe labeling 135 quality control process is described in more detail in reference to FIG. 4.

The target sample probes 130 may be labeled at one or more nucleotides during or after amplification. Labels suitable for use with microarray technology include labels detectable by spectroscopic, photochemical, biochemical, immunochemical, electrical, optical, or chemical means. Preferably, the detectable label is a luminescent label, such as fluorescent labels, chemiluminescent labels, bioluminescent labels, and colorimetric labels. Most preferably, the label is a fluorescent label such as fluorescein, rhodamine, lissamine, phycoerythrin, polymethine dye derivative, phosphor, and Cy2, Cy3, Cy3.5, Cy5, Cy5.5, Cy7. Commercially available fluorescent labels include fluorescein phosphoramidites such as Fluoreprime (Pharmacia, Piscataway, N.J.), Fluoredite (Millipore, Bedford, Mass.), and FAM (ABI, Foster City, Calif.). Other labels include biotin for staining with labeled streptavidin conjugate, magnetic beads (e.g., Dynabeads™), fluorescent dyes (e.g., texas red, rhodamine, green fluorescent protein), radiolabels (e.g., ³H, ¹²⁵I, ³⁵S, ¹⁴C, or ³²P), enzymes (e.g., horseradish peroxidase, alkaline phosphatase), and colorimetric labels such as colloidal gold or colored glass or plastic (e.g., polystyrene, polypropylene, latex) beads. See, e.g., U.S. Pat. Nos. 3,817,837; 3,850,752; 3,939,350; 3,996,345; 4,277,437; 4,275,149; and 4,366,241.

In a preferred embodiment, the target samples 130 may be fluorescently labeled or labeled with a radioactive isotope. For radioactive detection, a low energy emitter, such as ³³P-dCTP, is preferred due to close proximity of the oligonucleotides on the support. The fluorophores, Cy3-dUTP or Cy5-dUTP, may be used for fluorescent labeling 135. These fluorophores demonstrate efficient incorporation with reverse transcriptase and better yields. Furthermore, these fluorophores possess distinguishable excitation and emission spectra. Thus, two samples, each labeled with a different fluorophore, may be simultaneously hybridized to a microarray 110.

It is also advantageous to include quantitation controls within the target sample probes 140 to assure that amplification and labeling procedures do not change the true distribution of target sample probes in a sample. For this purpose, a sample may be spiked with a known amount of a control polynucleotide and the composition of polynucleotide sequences includes reference polynucleotide sequences which specifically hybridize with the control polynucleotides on the array. After hybridization and processing, the hybridization signals obtained should reflect accurately the amounts of control polynucleotide added to the sample.

The present invention relates to methods for performing quality control over the process of hybridizing the microarray 110 with the labeled target sample probes 140. A hybridization 142 quality control process may be performed to determine if the microarray 110 has been properly hybridized with the target sample probes. The hybridization 142 quality control process is described in more detail in reference to FIG. 4.

Specifically, labeled target sample probes 140 are hybridized 142 to the microarray 110. Hybridization causes a labeled target sample probe 140 and a complementary polynucleotide on the microarray 110 to form a stable duplex through base pairing. Hybridization methods are well known to those skilled in the art. See, e.g., Laboratory Techniques in Biochemistry and Molecular Biology, Vol. 24: Hybridization With Nucleic Acid Probes, P. Tijsson, ed. Elsevier, N.Y. (1993). Conditions can be selected for hybridization where only fully complementary target sample probes 140 and polynucleotides on the microarray 100 hybridize, i.e., each base pair must interact with its complementary base pair. Alternatively, conditions can be selected where target and polynucleotide sequences have mismatches but are still able to hybridize. Suitable conditions can be defined by salt concentration, temperature, and other chemicals and conditions well known in the art. Salt concentration may be less than about 750 mM NaCl and 75 mM trisodium citrate, preferably less than about 500 mM NaCl and 50 mM trisodium citrate, and most preferably less than about 250 mM NaCl and 25 mM trisodium citrate. Stringent temperature conditions will ordinarily include temperatures of at least about 22° C., more preferably of at least about 37° C., and most preferably of at least about 42° C. Varying additional parameters, such as hybridization time, the concentration of detergent or solvent, and the inclusion or exclusion of carrier DNA, are well known to those skilled in the art. Additional variations on these conditions will be readily apparent to those skilled in the art (Wahi, G. M. and S. L. Berger (1987) Methods Enzymol. 152: 399-407; Kimmel, A. R. (1987) Methods Enzymol. 152: 507-511; Ausubel, F. M. et al. (1997) Short Protocols in Molecular Biology, John Wiley & Sons, New York, N.Y.; and Sambrook, J. et al. (1989) Molecular Cloning, A Laboratory Manual, Cold Spring Harbor Press, Plainview, N.Y.).

More particularly, hybridization can be performed with buffers, such as 5×SSC/0.1%, SDS at 60° C. for about 6 hours. Subsequent washes are performed at higher stringency with buffers, such as 1×SCC/0.1% SDS at 45° C., then 0.1×SCC at 45° C. to retain hybridization of only those target/probe complexes that contain exactly complementary sequences.

Hybridization specificity can be evaluated by comparing the hybridization of specificity-control polynucleotides on the array to specificity-control polynucleotide probes that are added to the target sample probe in a known amount. The specificity-control target polynucleotides may have one or more sequence mismatches compared with the corresponding polynucleotide sequences. In this manner, whether only complementary target polynucleotides are hybridizing to the polynucleotide sequences or whether mismatched hybrid duplexes are forming is determined.

Hybridization reactions can be performed in absolute or differential hybridization formats. In the absolute hybridization format, target probes from one sample are hybridized to the sequences in a microarray format and signals detected after hybridization complex formation correlate to target probe levels in a sample. In the differential hybridization format, the differential expression of a set of genes in two biological samples is analyzed. For differential hybridization, target probes from both biological samples are prepared and labeled with different labeling moieties. A mixture of the two labeled target probes is added to a microarray. The microarray is then examined under conditions in which the emissions from the two different labels are individually detectable. Sequences in the microarray that are hybridized to substantially equal numbers of probes derived from both biological samples give a distinct combined fluorescence. PCT publication, WO 95/35505. In a preferred embodiment, the labels are fluorescent labels with distinguishable emission spectra, such as Cy3 and Cy5 fluorophores.

After hybridization, the microarray is washed to remove nonhybridized sample target probes 140 and complex formation between the hybridizable array oligonucleotides and the probes is detected. Methods for detecting complex formation are well known to those skilled in the art. In a preferred embodiment, the sample target probes 140 are labeled with a fluorescent label and measurement of levels and patterns of fluorescence indicative of complex formation is accomplished by fluorescence microscopy, preferably confocal fluorescence microscopy.

Typically, microarray fluorescence intensities can be normalized to take into account variations in hybridization intensities when more than one microarray is used under similar test conditions. In a preferred embodiment, individual polynucleotide target probe complex hybridization intensities are normalized using the intensities derived from internal normalization controls contained on each microarray.

The present invention further relates to a method of performing quality control over the process of washing a hybridized microarray. Specifically, a washing 144 quality control process may be performed to determine if the hybridized microarray has been properly washed. The washing 144 quality control process is described in more detail in reference to FIG. 4.

The present invention also relates to a method of performing quality control over the process of scanning a washed microarray to create an image 150. Specifically, a scanning 146 quality control process may be performed to determine if the washed microarray has been properly scanned. The scanning 146 quality control process is described in more detail in reference to FIG. 4.

The hybridized array 110 may then be subjected to laser excitation that produces an emission with a unique spectrum. The spectrum is scanned, for example, with a scanning confocal laser microscope generating monochrome images 150 of the microarray. These images 150 may be digitally processed and normalized based on a threshold value (e.g., background) using mathematical algorithms. For example, a threshold value of 0 may be assigned when no change in the level of fluorescence is observed; an increase in fluorescence may be assigned a value of +1 and a decrease in fluorescence may be assigned a value of −1. Normalization may be based on a designated subgroup of genes where variations in this subgroup are utilized to generate statistics applicable for evaluating the complete gene microarray. Chen et al., 2 J. BIOMED. OPTICS 364-67 (1997).

Methods for signal detection of labeled target probes hybridized to the microarray are well known in the art. For example, a radioactive labeled probe may be detected by radiation emission using photographic film or a gamma counter. For fluorescently labeled target nucleic acids, the localization of the label on the microarray may be accomplished with fluorescent microscopy. The hybridized microarray is excited with a light source at the excitation wavelength of the particular fluorescent label and the resulting fluorescence is detected 150. The excitation light source may be a laser appropriate for the excitation of the fluorescent label.

Confocal microscopy may be automated with a computer-controlled stage to automatically scan the entire microarray. Similarly, a microscope may be equipped with a phototransducer (e.g., a photomultiplier) attached to an automated data acquisition system to automatically record the fluorescence signal 150 produced by hybridization to oligonucleotide probes. See, e.g., U.S. Pat. No. 5,143,854.

Evaluation of the hybridization results may vary with the nature of the specific oligonucleotide probes used as well as the controls provided. For example, quantification of the fluorescence intensity 160 for each probe may be accomplished by measuring the probe signal strength at each location (representing a different probe) on the microarray (e.g., detection of the amount of florescence intensity produced by a fixed excitation illumination at each location on the array). The absolute intensities of the target probes hybridized to the microarray may then be compared with the intensities produced by the controls, providing a measure of the relative expression of the nucleic acids that hybridize to each of the oligonucleotides.

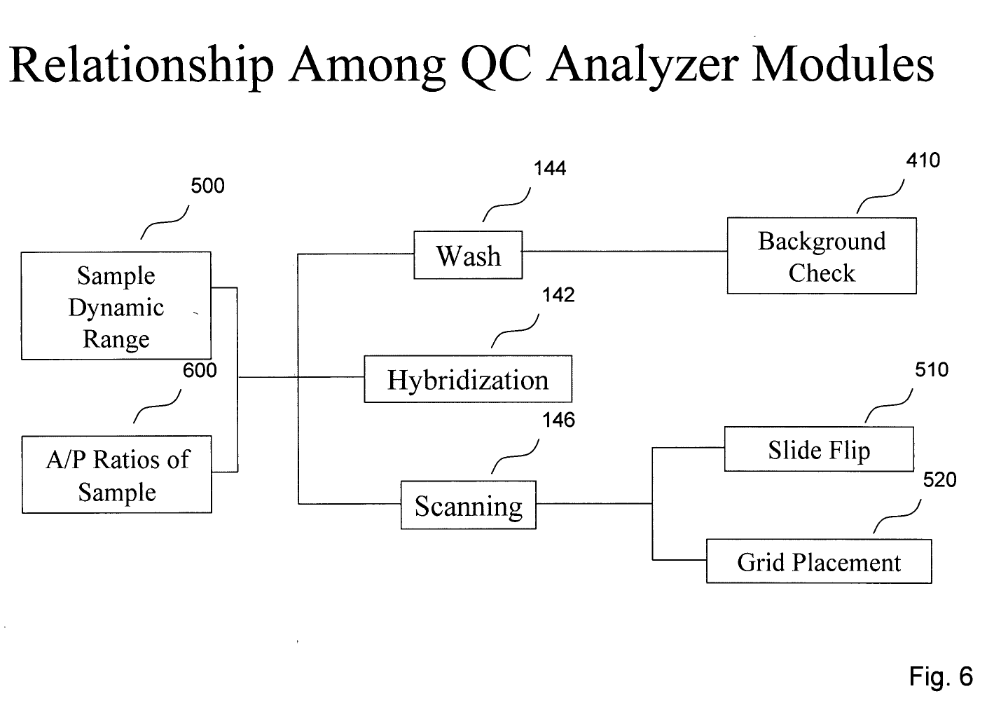

The present invention also relates to a method of performing quality control over the process of quantifying gene expression data 160 from an image 150. Specifically, a quantitation 155 quality control process may be performed to determine whether the retrieved gene expression data 160 conforms across multiple microarrays 110. The quantitation 155 quality control process is described in more detail in reference to FIG. 6.

Normalization of the signal derived from the target nucleic acids to the normalization controls may provide a control for variations in hybridization conditions. Typically, normalization may be accomplished by dividing the measured signal from the other probes in the array by the average signal produced by the normalization controls. Normalization may also include correction for variations due to sample preparation and amplification. Such normalization may be accomplished by dividing the measured signal by the average signal from the sample preparation/amplification control probes. The resulting values may be multiplied by a constant value to scale the results. Other methods for analyzing microarray data 160 are well-known in the art including coupled two-way clustering analysis, clustering algorithms (hierarchical clustering, self-organizing maps) and support vector machines. See, e.g., Eisen et al., 95 PROC. NATL. ACAD. SCI. USA 14863-68 (1998); Ermolaeva et al, 20 NATURE GENET. 19-23 (1998); Tamayo et al., 96 PROC. NATL. ACAD. SCI. USA 2907-12 (1999); Getz et al., 97 PROC. NATL. ACAD. SCI. USA 12079-84 (2000); Brown et al., 97 PROC. NATL. ACAD. SCI. USA 262-67 (2000); and Holter et al., 97 PROC. NATL. ACAD. SCI. USA 8409-14 (2000).

Control nucleic acid molecules (which includes oligonucleotides, individual RNA transcripts or other synthetic or naturally occurring species) corresponding to genomic DNA, housekeeping genes, or negative and positive control genes may also be present on the microarray 110. It is desirable that the selected oligonucleotides or transcripts for spiking into a control mix are not present in the genome of the study organism and have only minimal cross homology. Cross reactivity of putative spike-in sequences can be predicted electronically and confirmed empirically by hybridization. The ideal spike-in controls will quantitatively bind to their targets and add none or minimal background due to cross-reaction. These sequences may be used to calibrate background or basal level of expression or provide other useful information. These sequences may range from about 5 to about 50 nucleotides for oligonucleotides, whereas spiked in transcripts are in the 200-1000 nucleotide range. Examples of such control oligonucleotides or transcripts include genes commercially available from Stratagene (La Jolla, Calif.) (SpotReport®) derived from the green plant A. thaliana. These genes include Cab, RCA, rbcL, LTP4, LTP6, XCP2, RCP1, NAC1, TIM and PRKase. Other suitable transcripts commercially available also from Stratagene have been synthetically designed to have no homology to any known sequence, and are referred to as “Alien” sequences, and are sold commercially as SpotReport® Alien™. Using similar strategies non-homologous transcripts can be selected and used for this procedure the exact sequence is inconsequential as long as it hybridizes quantitatively and does not cross react. The addition of positive controls can include constitutively expressed housekeeping genes such as β-actin, ubiquitin G3PDH can also be useful in controlling experiments. However as these are dependent upon the sample and the tissue of origin their usefulness between tissue types and conditions is limited. Negative controls as their name implies are derived from sequences which do not cross react with the organism in question. A set of plant or alien transcripts can be used which are not spiked into the experiment. As no corresponding transcript should have been present in the original mix only background cross reactions for this spot should be observed

Expression level controls are oligonucleotides that hybridize specifically with constitutively expressed genes 130 in the biological sample 120 and are designed to control for the overall metabolic activity of a cell. Analysis of the variations in the levels of the expression control as compared to the expression level of the target probes indicates whether variations in expression level of a gene is due specifically to changes in transcription rate of that gene or to general variations in the health of the cell. Thus, if the expression levels of both the expression control and the target probes decrease or increase, these alterations may be attributed to changes in the metabolic activity of the cell as a whole, not to differential expression of the target gene in question. However, if only the expression of the target gene varies, then the variation in the expression may be attributed to differences in regulation of that gene and not to overall variations in the metabolic activity of the cell. Constitutively expressed genes such as housekeeping genes (e.g., β-actin gene, transferrin receptor gene, GAPDH gene) may serve as expression level controls.

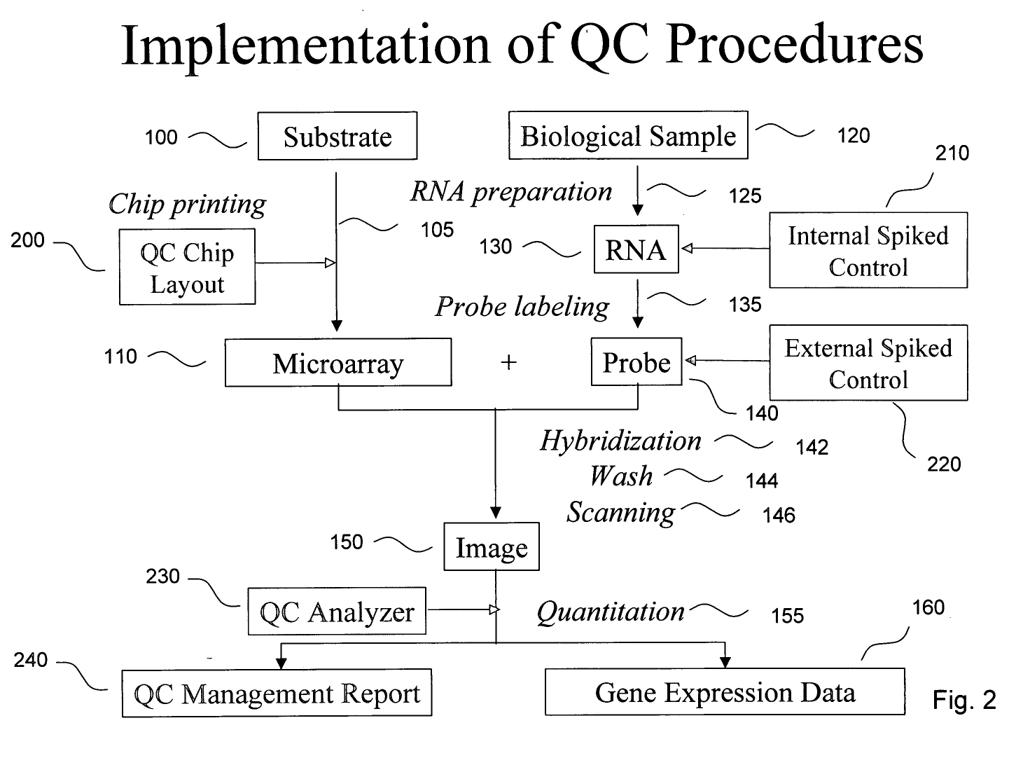

FIG. 2 depicts additional quality control functions that may be implemented by the present invention. QC Chip Layout 200 may create quality control points relating to the variation inherent in the chip printing process. These quality control points may be implemented by placing replicate spots on the microarray 110. Moreover, a dynamic range of values for RNA expression may be sought by placing spots of control oligonucleotides of different concentrations on the microarray 110. When the microarray is hybridized and imaged 150, the spots containing different concentrations of control oligonucleotides may produce different intensities of fluorescence. In addition, spots may be placed to retrieve other data inherent to other procedures including, but not limited to, RNA preparation, probe labeling, and hybridization. These spots may detect expression of different spiked controls. Indeed, it is advantageous to include quantitation controls within the sample to assure that amplification and labeling procedures do not change, for example, the true distribution of target probes in a sample. Hence, a sample may comprise a known amount of a control probes that specifically hybridize with control target oligonucleotide sequences on the array.

The Internal Spiked Control 210 process may seek to collect quality control data inherent to processes including, but not limited to, probe preparation and probe labeling. The Internal Spiked Control 210 process may be performed by placing a collection of pre-purified, high quality RNA of selected genes with known concentrations into the RNA population whose concentration is to be determined. This placement may occur before the probe labeling process. Both internal and external probes may be of RNA or DNA nature, depending upon labeling strategies used and in either case the spiked in RNA will be at a relative level as compared to the population of original experimental RNA sample. The following RNA products from A. thaliana obtained from Stratagene in custom format are spiked in as follows in each labeling reaction; RCP1 (10 ng), NAC1 (2 ng), TIM (0.4 ng), LTP4 (0.08 ng) and LTP6 (0.016 ng).

The External Spiked Control 220 process may seek to collect quality control data inherent to processes including, but not limited to, probe labeling and hybridization. The External Spiked Control 220 process may be performed by placing a collection of pre-purified, high quality prelabeled RNA (or DNA products representative of the original RNA) probes of selected genes with known concentrations into the RNA probe population whose concentration is to be determined. This placement may occur before the hybridization process. RNA from the following A. thaliana genes was obtained from a custom preparation from Stratagene to be prelabeled for in batch for addition to single experimental reactions at the following levels; PRKase (5 ng), XCP2 (1 ng) and Cab (0.2 ng). These products are labeled and quality controlled external to the primary reaction and added to each hybridization to provide a known hybridization control.

The QC Analyzer 230 process may analyze the gene expression data 160 from the image 150 of the hybridized microarray. The functions of the QC Analyzer 230 are described below in reference to FIG. 4. The QC Management Report 240 process may create a report summarizing the quality control data analyzed by the QC Analyzer 230.

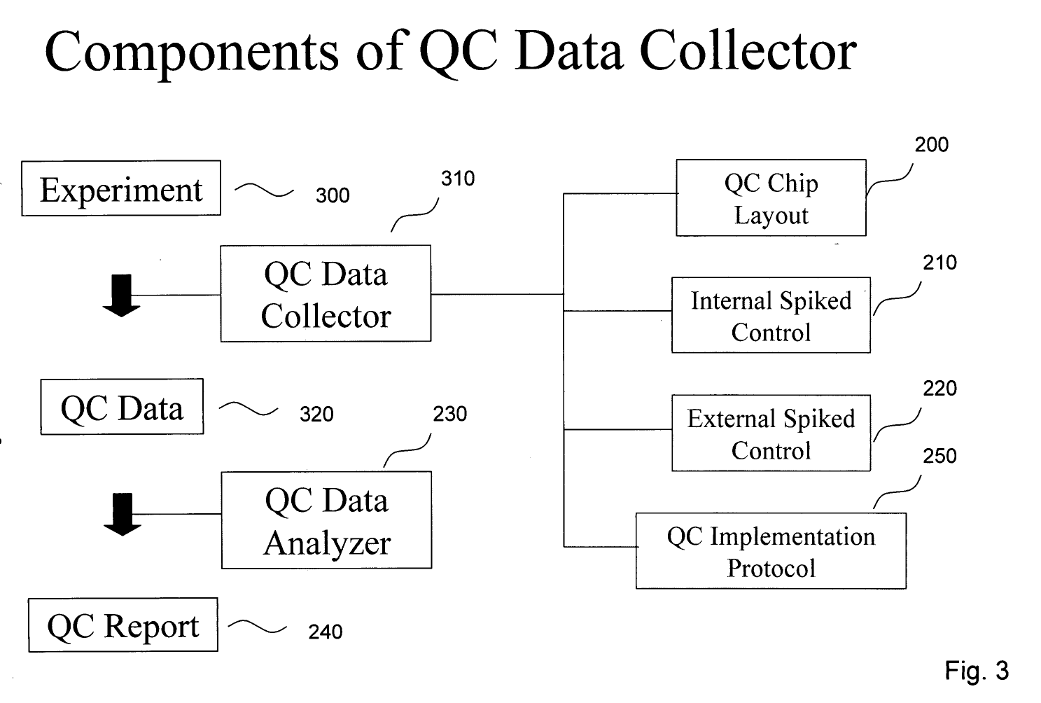

FIG. 3 depicts a process flow that may be used to implement the present invention. In FIG. 3, an Experiment 300 may be performed, data may be collected by a QC Data Collector 310, QC Data 320 may be passed to a QC Data Analyzer 230, and a QC Report 240 may be generated by the QC Data Analyzer 230. In this process, the QC Data Collector 310 process may perform one or more functions including, but not limited to, QC Chip Layout 200, Internal Spiked Control 210, and External Spiked Control 220, which are described above in reference to FIG. 2. The QC Data Collector 310 may, for example, apply the QC Implementation Protocol 250 in collecting the data.

FIG. 4 depicts a process flow of FIG. 3 that may be used to implement the present invention. FIG. 4 further shows a preferred embodiment for the QC Data Analyzer 230. In FIG. 4, an Experiment 300 may be performed, data may be collected by a QC Data Collector 310, QC Data 320 may be passed to a QC Data Analyzer 230, and a QC Report 240 may be generated by the QC Data Analyzer 230. In the embodiment of the present invention, the QC Data Analyzer 230 may perform two operations: Failure Detection 400 and Data Consistency 420. Failure Detection 400 may include quality control processes including, but not limited to, Chip Printing 105, RNA Preparation 125, Probe Labeling 135, Hybridization 142, Washing 144, Background Check 410, and Scanning 146. Data Consistency 420 may include quality control processes including, but not limited to, Reproducibility 422 and Outlier Detection 424. The quality control processes associated with Failure Detection 400 may relate to examining results generated by testing a microarray individually. The quality control processes associated with Data Consistency 420 may relate to examining results from a plurality of microarrays in conjunction with each other.

The Chip Printing 105 process may collect quality control data including, but not limited to, variations of expression data in replicate spots. The Chip Printing 105 process may then perform an algorithm upon the collected data. In an embodiment of the present invention, the algorithm may consist of performing a logarithmic transformation of the gene expression intensity data of all spots, calculating the variation of the log-transformed gene expression intensity data for each spot from the median, determining the distribution of the variation of the gene expression intensity data from all spots, comparing the variation distribution with a pre-defined distribution pattern, and calculating the percentage of spots that abnormally vary from the pre-defined distribution pattern. The pre-defined distribution pattern may be derived from the distribution patterns of a set of microarrays created with a normal chip printing process.

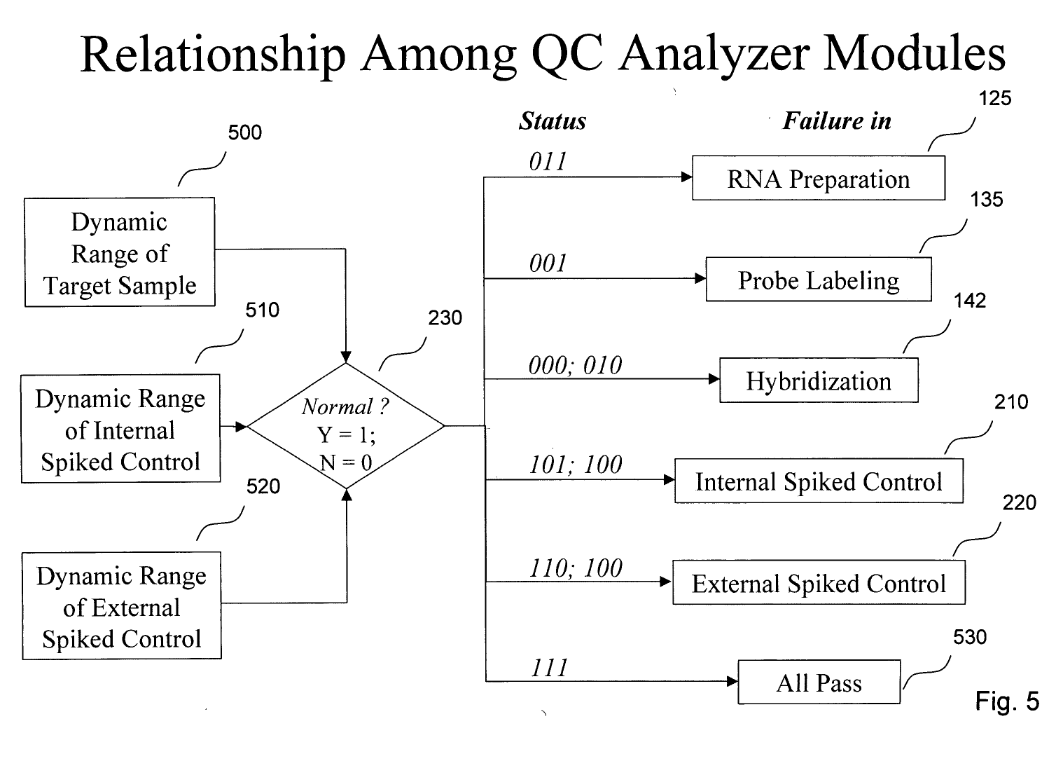

In a preferred embodiment of the present invention, the quality control processes of RNA Preparation 125, Probe Labeling 135, and Hybridization 142 may utilize the analysis of three ranges of intensity data as depicted in FIG. 5. The three ranges of data are the Dynamic Range of Sample 500, the Dynamic Range of Internal Spiked Control 510, and the Dynamic Range of External Spiked Control 520. Whether the gene expression intensity data for all spots falls within the Dynamic Range of Sample 500, the gene expression intensity data for internal spiked control spots falls within the Dynamic Range of Internal Spiked Control 510, and the gene expression intensity data for external spiked control spots falls within the Dynamic Range of External Spiked Control 520 may determine the type of failure that occurred, if one exists.

A microarray may be determined to be bounded by the Dynamic Range of Sample 500 by collecting the gene expression intensity data for all spots in the microarray, performing a logarithmic transformation on the gene expression intensity data at each spot, calculating the mean of the gene expression intensity data and the standard deviation of the gene expression intensity data, converting each log-transformed intensity value into a Z-score, comparing a set of percentiles of Z-scores to a set of pre-defined values, and calculating a concordance correlation. If a microarray is not bounded by the Dynamic Range of Sample 500, a flag denoting a failure may be set in the QC Data Analyzer 230. The set of pre-defined values may be derived from a set of normal dynamic ranges for RNA samples. The Z-score value for a spot may be computed by the following equation:

(Zₛₚₒₜ=[Intₛₚₒₜ−Mean]/Stddev) (Eqn. 1)

A microarray may be determined to be bounded by the Dynamic Range of Internal Spiked Control 510 by collecting the gene expression intensity data for all internal spiked control spots in the microarray, performing a logarithmic transformation on the gene expression intensity data at each internal spiked control spot, calculating the mean of the gene expression intensity data and the standard deviation of the gene expression intensity data, converting each log-transformed intensity value into a Z-score, comparing a set of percentiles of Z-scores to a set of pre-defined values, and calculating a concordance correlation. If a microarray is not bounded by the Dynamic Range of Internal Spiked Control 510, a flag denoting a failure may be set in the QC Data Analyzer 230. The set of pre-defined values may be derived from a set of normal dynamic ranges for internal spiked control spots for RNA samples.

A microarray may be determined to be bounded by the Dynamic Range of External Spiked Control 520 by collecting the gene expression intensity data for all external spiked control spots in the microarray, performing a logarithmic transformation on the gene expression intensity data at each external spiked control spot, calculating the mean of the gene expression intensity data and the standard deviation of the gene expression intensity data, converting each log-transformed intensity value into a Z-score, comparing a set of percentiles of Z-scores to a set of pre-defined values, and calculating a concordance correlation. If a microarray is not bounded by the Dynamic Range of External Spiked Control 520, a flag denoting a failure may be set in the QC Data Analyzer 230. The set of pre-defined values may be derived from a set of normal dynamic ranges for external spiked control spots for RNA samples. The All Pass status 530, indicates that no failure was observed.

Referring to FIG. 4, the RNA Preparation 125 process may collect quality control data including, but not limited to, the dynamic range of a prepared sample, the dynamic range of internal spiked controls, and dynamic range of external spiked controls for a microarray. The RNA Preparation 125 process may then perform an algorithm upon the collected data. In an embodiment of the present invention, the algorithm may consist of calculating the ratio of the dynamic range of a prepared RNA sample over the dynamic range of internal spiked controls and comparing the ratio to a pre-defined value. The pre-defined value may be derived from a set of normal RNA samples.

The Probe Labeling 135 process may collect quality control data including, but not limited to, the dynamic range of a prepared sample, the dynamic range of internal spiked controls, and dynamic range of external spiked controls for a DNA microarray. The Probe Labeling 135 process may then perform an algorithm upon the collected data. In an embodiment of the present invention, the algorithm may consist of calculating the ratio of the dynamic range of a prepared sample over the dynamic range of external spiked controls, calculating the ratio of the dynamic range of internal spiked controls over the dynamic range of external spiked controls, and comparing both ratios to pre-defined values. The pre-defined values may be derived from a set of normal RNA probes.

The Hybridization 142 process may collect quality control data including, but not limited to, the dynamic range of a prepared sample, the dynamic range of internal spiked controls, and dynamic range of external spiked controls for a microarray. The Hybridization 142 process may then perform an algorithm upon the collected data. In an embodiment of the present invention, the algorithm may consist of calculating the ratio of the dynamic range of a prepared sample over the dynamic range of external spiked controls, calculating the ratio of the dynamic range of internal spiked controls over the dynamic range of external spiked controls, and comparing both ratios to a pre-defined value. The pre-defined value may be derived from a set of normal samples with normal probe labeling and normal hybridization.

In a preferred embodiment of the present invention, the Scanning quality control process 146 may utilize the analysis of two data sets as depicted in FIG. 6. The two data sets are the Dynamic Range of Sample 500 and the Absence/Presence Ratios of Sample 600.

The Background Check 410 process may collect quality control data including, but not limited to, the background intensity data of all spots. The Background Check 410 process may then perform an algorithm upon the collected data. In an embodiment of the present invention, the algorithm may consist of calculating the mean and standard deviation for the set of background intensities of all spots, converting the background intensity for each spot into a Z-score value, and calculating the percentage of spots for which the absolute Z-score is greater than one or more threshold values. In a preferred embodiment, the one or more threshold values may include three threshold values equal to, for example, 1, 2, and 3.

The computation of the Absence/Presence Ratios of an RNA Sample 600 may be implemented by collecting the intensity data and the background intensity data for all spots and performing an algorithm on the collected data. In a preferred embodiment of the present invention, the algorithm may consist of calculating the median and standard deviation of the gene expression intensity data for the good portion of the chip background; determining whether a spot is present or absent; calculating a presence ratio; and calculating a determination ratio. In a preferred embodiment, the good portion of the chip background may include all spots that are not classified as outliers by the QC Data Analyzer 230. In a preferred embodiment, a spot may be determined to be present if the gene expression intensity of the spot is greater than the median intensity of the background by a pre-defined value and the gene expression intensity of the spot is greater than the intensity of the immediate background. In a preferred embodiment, the pre-defined value may equal, for example, twice the standard deviation of the intensity. In a preferred embodiment, a spot may be determined to be absent if the intensity of the spot is less than the median of the background intensity. In a preferred embodiment, the presence ratio and determination ratios may be calculated by the following equations:

Presence Ratio=N(Present)/[N(Present)+N(Absent)] (Eqn. 2)

Determination Ratio=[N(Present)+N(Absent)]/N(Spots) (Eqn. 3)

The Scanning process 146 may be further sub-divided into the Slide Flip 510 and the Grid Placement 520 processes.

The Slide Flip 510 process may collect quality control data including, but not limited to, the gene expression intensity data for all control spots including, without limitation, external spikes, internal spikes, blank spots, etc. The Slide Flip 510 process may then perform an algorithm upon the collected data. In a preferred embodiment, the algorithm may consist of comparing the gene expression intensity for each individual control spot with a pre-defined intensity range and determining the percentage of control spots that do not fall within the normal intensity range.

The Grid Placement 520 process may collect quality control data including, but not limited to, gene expression intensity data from the microarray and PositionOff data retrieved from a software tool, such as Imagene. The Grid Placement 520 process may then perform an algorithm upon the collected data. In a preferred embodiment, the algorithm may consist of calculating the mean and standard deviation of the gene expression intensity values for the entire chip, calculating the mean of the gene expression intensity value for each row of a microarray, calculating the mean of the gene expression intensity value for each column of a microarray, converting each calculated mean to a Z-score related to the mean and standard deviation of the entire microarray, comparing the Z-score for each row mean to a pre-defined value, comparing the Z-score for each column mean to a pre-defined value, and calculating the percentage of Z-scores greater than a pre-defined value. The pre-defined value to which the Z-score for each row mean is compared may be different or equal to the pre-defined value to which the Z-score for each column mean is compared. In a preferred embodiment, both pre-defined values may be equal to, for example, 4.

The Reproducibility process 422 may be sub-divided into the Data Correlation process and the Data Consistency process.

The Data Correlation process may collect quality control data from multiple microarrays including, but not limited to, gene expression intensity data for all oligonucleotides hybridized by the same probe. The Data Correlation process may then implement an algorithm on the collected data. In a preferred embodiment, the algorithm may consist of concatenating each group of intensity values from spots at a particular location into vectors, calculating the Pearson correlation coefficient between each set of two vectors, calculating a concordance correlation value between each set of two vectors, comparing the Pearson correlation coefficient with a pre-defined value derived from chips possessing normal consistency, and comparing the concordance correlation value with a pre-defined value derived from chips possessing normal consistency. In a preferred embodiment, replicate spots may be considered as separate replicate elements in two or more separated vectors.

The Data Consistency process may collect quality control data from microarrays including, but not limited to, gene expression intensity data for all spots hybridized by the same probe. The Data Consistency process may then implement an algorithm on the collected data. In a preferred embodiment, the algorithm may consist of performing a logarithmic transformation of the gene expression intensity data at each spot; calculating the mean, standard deviation and CV of the log-transformed intensity data for replicate spots associated with a particular gene; calculating the average CV of all genes for each panel, and comparing the panel average CV to a pre-defined value. In a preferred embodiment, replicate spots associated with a particular gene may be considered together for the purpose of computing the mean, standard deviation and CV of the log-transformed intensity data regardless of whether they are on the same DNA microarray. In a preferred embodiment, the pre-defined value against which the panel-average CV is compared may be, for example, 20.

The Outlier Detection 424 process may collect quality control data from three or more DNA microarrays including, but not limited to, gene expression intensity data for all spots on DNA microarrays which are hybridized by the same probe and morphology parameters for each spot from a software tool, such as Imagene 4.2 from BioDiscovery Inc. (Marina del Rey, Calif.). The morphology data may include, but is not limited to, the mode of the background intensity, the standard deviation of the background intensity, the mean of the background intensity, the mode of the signal intensity, the standard deviation of the signal intensity, the mean of the signal intensity, the median of the signal intensity, the area of the signal intensity, the area of an ignored section, the median of the intensity of the ignored section, and a PositionOff value. Each of the morphology parameters may be provided by the software tool for each spot hybridized with a unique probe.

The Outlier Detection 424 process may then implement an algorithm on the collected data. In a preferred embodiment, the algorithm may consist of performing a logarithmic transformation on the gene expression intensity data of replicate spots; calculating the CV, median, mean, and standard deviation of the log-transformed replicate spot gene expression intensity data; calculating, for genes with CV greater than a pre-defined value, such as 30, morphology information for each individual replicate spot based on the collected data; and flagging a spot intensity as an outlier if the score for its morphology information calculations is greater than a pre-defined value, such as 1. In a preferred embodiment, the morphology information calculations may include, without limitation, a signal intensity ratio, a background Z-score value, a signal CV, a background CV, an ignored area ratio, an ignored median ratio, a Q signal area, and a PositionOff Z-score value.

In a preferred embodiment, the calculation for the signal intensity ratio may consist of dividing the gene expression intensity of a spot by the median gene expression intensity for all spots; adding a pre-defined value, such as 1, to the score if the ratio is greater than a pre-defined value, such as 1.4; and adding a pre-defined value, such as 1, to the score if the ratio is less than a pre-defined value, such as 0.714.

In a preferred embodiment, the calculation for the background Z-score value may consist of converting the mode of the background intensity into a Z-score value and adding a pre-defined value, such as 0.5, to the score if the Z-score value is greater than a pre-defined value, such as 3.

In a preferred embodiment, the calculation for the signal CV may consist of dividing the standard deviation of the signal intensity by the mean of the signal intensity and adding a pre-defined value, such as 1, to the score if the signal CV is greater than a pre-defined value, such as 40, and the logarithmic transformation of the mode of the signal intensity is less than a pre-defined value, such as 3.7.

In a preferred embodiment, the calculation for the background CV may consist of dividing the standard deviation of the background intensity by the mean of the background intensity and adding a pre-defined value, such as 1, to the score if the background CV is greater than a pre-defined value, such as 40, and the logarithmic transformation of the mode of the background intensity is less than a pre-defined value, such as 3.7.

In a preferred embodiment, the calculation for the ignored area ratio may consist of dividing the area of the ignored section by the area of the signal intensity and adding a pre-defined value to the score if the ignored area ratio is greater than a pre-defined value.

In a preferred embodiment, the calculation for the ignored median ratio may consist of dividing the median of the intensity of the ignored section by the mode of the signal intensity and adding a pre-defined value, such as 1, to the score if the ignore median ratio is greater than a pre-defined value, such as 6.

In a preferred embodiment, the calculation for the Q signal area may consist of the following formula:

Q Signal Area=e^{-|A-Ao|/Ao} (Eqn. 4)

where Aₒ is the average of the signal area of the whole chip and A is the area of the whole chip. The calculation may further consist of adding a pre-defined value, such as 0.5, to the score if the Q signal area is greater than a pre-defined value, such as 0.51.

In a preferred embodiment, the calculation for the PositionOff Z-score value may consist of converting the PositionOff value into a Z-score value and adding a pre-defined value, such as 0.5, to the score if the PositionOff Z-score value is greater than a pre-defined value, such as 5.

Various modifications and variations of the described methods and systems of the invention will be apparent to those skilled in the art without departing from the scope and spirit of the invention. Although the invention has been described in connection with preferred embodiments, it should be understood that the invention as claimed should not be unduly limited to such preferred embodiments. Indeed, various modifications of the described modes for carrying out the invention which are obvious to those skilled in molecular biology or related fields are intended to be within the scope of the following claims.

The disclosures of all references and publications cited above are expressly incorporated by reference in their entireties to the same extent as if each were incorporated by reference individually.

We claim:

1. A method of performing quality control in gene expression profiling on a microarray by determining a variation between a desired printing of a microarray and an actual printing of a microarray comprising:
retrieving gene expression data from one or more replicate spots;
performing a logarithmic transformation on said gene expression data from each of said one or more replicate spots;
calculating variations between said log-transformed gene expression data and an expected value for each of said one or more replicate spots;
determining a distribution of said variations for each of said one or more replicate spots;
comparing said distribution with a pre-defined distribution; and
calculating a percentage of said one or more replicate spots for which said variation exceeds a threshold.

2. A method of performing quality control in gene expression profiling on a microarray during target sample preparation from a biological sample comprising:
generating a dynamic range of values for a target sample;
generating a dynamic range of values for internal spiked controls;
calculating a ratio between said dynamic range of values for said target sample and said dynamic range of values for internal spiked controls; and
comparing said ratio to a pre-defined value.

3. A method of performing quality control in gene expression profiling on a microarray during labeling of target samples comprising:
generating a dynamic range of values for a labeled target sample;
generating a dynamic range of values for internal spiked controls;
generating a dynamic range of values for external spiked controls;
calculating a first ratio between said dynamic range of values for said labeled target sample and said dynamic range of values for internal spiked controls;
comparing said first ratio to a first pre-defined value;
calculating a second ratio between said dynamic range of values for internal spiked controls and said dynamic range of values for external spiked controls; and
comparing said second ratio to a second pre-defined value.

4. A method of performing quality control in gene expression profiling on a microarray during hybridization of said microarray and a labeled target sample comprising:
generating a dynamic range of values for said labeled target sample;
generating a dynamic range of values for internal spiked controls;
generating a dynamic range of values for external spiked controls;
calculating a first ratio between said dynamic range of values for said labeled target sample and said dynamic range of values for internal spiked controls;
comparing said first ratio to a first pre-defined value;
calculating a second ratio between said dynamic range of values for internal spiked controls and said dynamic range of values for external spiked controls; and
comparing said second ratio to a second pre-defined value.

5. The method of claim 4, wherein an internal spiked control error flag is set when said first ratio is substantially equal to said first pre-defined value and said second ratio is less than said second pre-defined value.

6. The method of claim 4, wherein an external spiked control flag is set when said first ratio is greater than said first pre-defined value and said second ratio is greater than said second pre-defined value.

7. A method of performing quality control in gene expression profiling on a microarray during a background intensity check of said microarray hybridized with a labeled target sample comprising:
retrieving intensity data from one or more replicate spots;
calculating a mean for said intensity data from said one or more replicate spots;
calculating a standard deviation for said intensity data from said one or more replicate spots;
generating a Z-score transformation for each of said one or more replicate spots from said intensity data from each of said one or more replicate spots, wherein said Z-score transformation for a particular replicate spot is computed by subtracting said mean for said intensity data from an intensity of a replicate spot and dividing by said standard deviation for said intensity data;
calculating a first percentage of spots for which the absolute value of said Z-score exceeds 1;
comparing said first percentage with a pre-defined value;
calculating a second percentage of spots for which the absolute value of said Z-score exceeds 2;
comparing said second percentage with a pre-defined value;
calculating a third percentage of spots for which the absolute value of said Z-score exceeds 3; and
comparing said third percentage with a pre-defined value.

8. A method of performing quality control in gene expression profiling on a microarray during scanning of said microarray hybridized with a labeled target sample comprising the steps of:
slide flipping; and
grid placement.

9. The method of claim 8, wherein said slide flipping step comprises:
retrieving intensity data for one or more replicate spots;
comparing said intensity data for each of said one or more replicate spots with a pre-defined normal intensity range; and
determining a percentage of said one or more replicate spots not within said pre-defined normal intensity range.

10. The method of claim 8, wherein said grid placement step comprises:
retrieving intensity data from one or more replicate spots;
calculating a first mean for said intensity data from said one or more replicate spots;
calculating a standard deviation for said intensity data from said one or more replicate spots;
calculating a second mean for said intensity data from each row of oligonucleotides of said microarray;
generating a first Z-score transformation for each second mean, wherein said Z-score transformation for a particular row is computed by subtracting said first mean from said second mean for a particular row and dividing by said standard deviation for said intensity data;
calculating a third mean for each column of said microarray;
generating a second Z-score transformation for each third mean, wherein said Z-score transformation for a particular column is computed by subtracting said first mean from said third mean for a particular column and dividing by said standard deviation for said intensity data;
calculating a percentage of said first Z-score transformations and said second Z-score transformations that are greater than a pre-defined value.

11. The method of claim 10, wherein said pre-defined value is 4.

12. A method of performing quality control in gene expression profiling on a microarray during quantitation of an image of said microarray hybridized with a labeled target sample comprising:
retrieving intensity data for one or more genes each containing one or more replicate spots;
generating log-transformed intensity data by performing a logarithmic transformation on said intensity data for each of said one or more replicate spots;
retrieving a set of parameters from Imagene for each of said one or more replicate spots;
calculating a mean for said log-transformed intensity data for each of said one or more genes;
calculating a standard deviation for said log-transformed intensity data for each of said one or more genes;
calculating a CV for said log-transformed intensity data for each of said one or more genes; and
determining outlier spots when said CV is greater than a pre-defined value.

13. The method of claim 12, wherein said pre-defined value is 30.

14. The method of claim 12, wherein said determining outlier spots comprises:
calculating one or more metrics for each of said one or more replicate spots based upon said set of parameters and said logarithmic transformation of said intensity data;
computing an outlier score; and
marking a replicate spot as an outlier if said outlier score exceeds a pre-defined value.

15. The method of claim 14, wherein said pre-defined value is 1.

16. The method of claim 14, wherein said set of parameters comprises one or more of the following:
background mode;
background standard deviation;
background mean;
signal mode;
signal standard deviation;
signal mean;
signal median;
signal area;
ignored area;
ignored median; and
a PositionOff value.

17. The method of claim 16, wherein said one or more metrics comprise one or more of the following:
a spot intensity ratio, wherein said spot intensity ratio is computed by dividing an intensity for a replicate spot by said signal median for said replicate spot;
a Z-score transformation of said background mode, wherein said Z-score transformation for said background mode is computed by subtracting said background mean from said background mode and dividing by said background standard deviation;
a signal CV, wherein said signal CV is computed by dividing said signal standard deviation by said signal mean;
a background CV, wherein said background CV is computed by dividing said background standard deviation by said background mean;
an ignored area ratio, wherein said ignored area ratio is computed by dividing said ignored area by said signal area;
an ignored median ratio, wherein said ignored median ratio is computed by dividing said ignored median by said signal mode;
a Q signal area value, wherein said Q signal area value is equal to e^{-|A-Ao|/Ao}, wherein Ao is an average of said signal area for said one or more genes; and
a Z-score transformation of said PositionOff value.

18. The method of claim 17, wherein said computing an outlier score comprises one or more of the following:
setting said outlier score to 0;
adding a first outlier value to said outlier score when said spot intensity ratio is greater than a first pre-defined value;
adding a second outlier value to said outlier score when said spot intensity ratio is less than a second pre-defined value;
adding a third outlier value to said outlier score when said Z-score transformation of said background mode is greater than a third pre-defined value;
adding a fourth outlier value to said outlier score when said signal CV is greater than a fourth pre-defined value and a logarithmic transformation of said signal mode is less than a fifth pre-defined value;
adding a fifth outlier value to said outlier score when said background CV is greater than a sixth pre-defined value and a logarithmic transformation of said signal mode is less than a seventh pre-defined value;
adding a sixth outlier value to said outlier score when said Q signal area is less than an eighth pre-defined value;
adding a seventh outlier value to said outlier score when said ignored median ratio is greater than a ninth pre-defined value; and
adding an eighth outlier value to said outlier score when said Z-score transformation of said PositionOff value is greater than a tenth pre-defined value.

19. The method of claim 18, wherein said first outlier value is 1.

20. The method of claim 18, wherein said first pre-defined value is 1.4.

21. The method of claim 18, wherein said second outlier value is 1.

22. The method of claim 18, wherein said second pre-defined value is 0.714.

23. The method of claim 18, wherein said third outlier value is 0.5.

24. The method of claim 18, wherein said third pre-defined value is 3.

25. The method of claim 18, wherein said fourth outlier value is 1.

26. The method of claim 18, wherein said fourth pre-defined value is 40.

27. The method of claim 18, wherein said fifth pre-defined value is 3.7.

28. The method of claim 18, wherein said fifth outlier value is 1.

29. The method of claim 18, wherein said sixth pre-defined value is 40.

30. The method of claim 18, wherein said seventh pre-defined value is 3.7.

31. The method of claim 18, wherein said sixth outlier value is 0.5.

32. The method of claim 18, wherein said eighth pre-defined value is 0.51.

33. The method of claim 18, wherein said seventh outlier value is 1.

34. The method of claim 18, wherein said ninth pre-defined value is 6.

35. The method of claim 18, wherein said eighth outlier value is 0.5.

36. The method of claim 18, wherein said tenth pre-defined value is 5.
